(19) United States
(12) Patent Application Publication
(10) Pub. No.: US 2009/0186347 A1
(43) Pub. Date: Jul. 23, 2009

(54) Markers for metabolic syndrome

(76) Inventors: David R. Cox, Belmont, CA (US); Kelly A. Frazer, San Mateo, CA (US); David A. Hinds, Sunnyvale, CA (US); Craig L. Hyde, Old Lyme, CT (US); John F. Thompson, Warwick, RI (US)

Correspondence Address: Goodwin Procter LLP;Attn: Patent Administrator, 135 Commonwealth Drive, Menlo Park, CA, 94025-1105, US

(21) Appl. No.: 12/152,141

(22) Filed: May 9, 2008

Related U.S. Application Data
(60) Provisional application No. 60/930,033, filed on May 11, 2007.

(51) Int. Cl. C12Q 1/68

(52) U.S. Cl. 435/6

(57) ABSTRACT

Correlations between polymorphisms and metabolic syndrome, insulin resistance, obesity, high blood pressure, dyslipidemia, diabetes and/or myocardial infarction are provided. Methods of diagnosing and treating metabolic syndrome, insulin resistance, obesity, high blood pressure, dyslipidemia, diabetes and/or myocardial infarction are provided. Systems and kits for diagnosis and treatment of metabolic syndrome, insulin resistance, obesity, high blood pressure, dyslipidemia, diabetes and/or myocardial infarction are provided.

### CROSS-REFERENCE TO RELATED APPLICATION

This application is a non-provisional application filed under 37 CFR 1.53(b)(1), claiming priority under 35 USC 119(e) to provisional application No. 60/930,033 filed May 11, 2007, the contents of which are incorporated herein by reference.

### BACKGROUND OF THE INVENTION

Metabolic syndrome is a collection of health disorders or risks that increase the chance of developing heart disease, stroke, and diabetes. The condition is also known by other names, including Syndrome X, insulin resistance syndrome, and dysmetabolic syndrome. Metabolic syndrome can include any of a variety of underlying metabolic phenotypes, including insulin resistance and/or obesity predisposition phenotypes.

Metabolic syndrome is often characterized by any of a number of metabolic disorders or risk factors, which are generally considered to most typify metabolic syndrome when more than one of these factors are present in a single individual. The factors include: central obesity (disproportionate fat tissue in and around the abdomen), atherogenic dyslipidemia (these include a family of blood fat disorders including, e.g., high triglycerides, low HDL cholesterol, and high LDL cholesterol that can foster plaque buildups in the vascular system, including artery walls), high blood pressure (130/85 mmHg or higher), insulin resistance or glucose intolerance (the inability to properly use insulin or blood sugar), a chronic prothrombotic state (e.g., characterized by high fibrinogen or plasminogen activator inhibitor [−1] levels in the blood), and a chronic proinflammatory state (e.g., characterized by higher than normal levels of high-sensitivity C-reactive protein in the blood). People with metabolic syndrome are at increased risk of coronary heart disease, other diseases related to plaque buildups in artery walls (e.g., stroke and peripheral vascular disease) and Type 2 Diabetes.

Metabolic syndrome is extremely common, particularly in the United States, where roughly 50 million people are thought to have the disorder. Roughly one in five Americans has metabolic syndrome. The number of people with metabolic syndrome increases with age, affecting more than 40 percent of people in their 60s and 70s. The underlying causes of metabolic syndrome are, in many respects, quite unclear-though certain effects of the disorder such as obesity and lack of physical activity are often causal in nature as well. Given inheritance patterns for the disorder, there also appear to be genetic factors that underlie the syndrome.

For example, some people with metabolic syndrome are genetically predisposed to insulin resistance, which typically leads to obesity. On the other hand, obesity can and does also elicit insulin resistance. Thus, while it is true that most people with insulin resistance have central obesity, it is not always clear whether insulin resistance causes central obesity or whether central obesity causes insulin resistance. The underlying biological mechanism(s) between insulin resistance and metabolic risk factors (at the molecular level) are not fully understood and are also likely to be quite complex.

Not only is metabolic syndrome likely a result of several interacting genetic and environmental factors, but the criteria for diagnosing metabolic syndrome are somewhat variable. Criteria considered most relevant by the “Third Report of the National Cholesterol Education Program (NCEP) Expert Panel on Detection, Evaluation, and Treatment of High Blood Cholesterol in Adults (Adult Treatment Panel III)” in the diagnosis of metabolic disorder provide one widely used current set of diagnostic criteria.

Under the NCEP criteria, metabolic syndrome can be clinically identified by presence of three or more of the following components in a single patient: (1) central obesity, as measured by waist circumference (women with a waist circumference greater than 35 inches; for men greater than 40 inches); (2) fasting blood triglycerides greater than or equal to 150 mg/dL; (3) blood HDL cholesterol (for women less than 50 mg/dL, for men less than 40 mg/dL); (4) blood pressure greater than or equal to 130/85 mmHg; and (5) fasting glucose greater than or equal to 110 mg/dL. Other features such as insulin resistance (e.g., increased fasting blood insulin), prothrombotic state or proinflammatory state are not generally required for clinical diagnosis, though they are certainly also indicative of metabolic syndrome and follow-up studies on these attributes can be used to further confirm diagnosis of metabolic syndrome. For example, insulin resistance, even in the absence of the NCEP criteria, is often indicative of metabolic syndrome.

Treatment for metabolic syndrome, obesity, insulin resistance, high blood pressure, dyslipidemia, etc., can include a variety of clinical approaches, including weight loss and exercise (these two safest and most effective treatments are also often quite difficult to achieve in practice), and dietary changes. These dietary changes include: maintaining a diet that limits carbohydrates to 50 percent or less of total calories; eating foods defined as complex carbohydrates, such as whole grain bread (instead of white), brown rice (instead of white), sugars that are unrefined, increasing fiber consumption by eating legumes (for example, beans), whole grains, fruits and vegetables, reducing intake of red meats and poultry, consumption of “healthy” fats, such as those in olive oil, flaxseed oil and nuts, limiting alcohol intake, etc. In addition, treatment of blood pressure, and blood triglyceride levels can be controlled by a variety of available drugs (e.g., cholesterol modulating drugs), as can clotting disorders (e.g., via aspirin therapy) and in general, prothrombotic or proinflammatory states. If metabolic syndrome leads to diabetes, there are, of course, many treatments available for this disease, including those noted above, in conjunction with insulin treatment.

Thus, while there are a variety of strategies for treatment of metabolic syndrome, obesity predisposition, insulin resistance, high blood pressure, dyslipidemia, etc., such as diet and exercise, drug therapy, etc., the molecular basis for these disorders is not clear, making diagnosis or prognosis of these metabolic disorders problematic and the design of therapeutic agents to treat them quite difficult.

Thus, while a considerable amount is known about metabolic syndrome at the clinical level, disease diagnosis for this central human disease and its related disorders is relatively imprecise, and early detection of susceptible individuals is difficult. The present invention provides a number of new genetic correlations between metabolic syndrome (including e.g., obesity predisposition, insulin resistance, high blood pressure, and dyslipidemia), etc., and various polymorphic alleles, providing the basis for improved diagnosis of disease, early detection of susceptible individuals (e.g., before metabolic syndrome is clinically manifested), targets for potential disease modulators, as well as an improved understanding of metabolic syndrome and its related disorders at the molecular and cellular level. These and other features of the invention will be apparent upon review of the following.

### SUMMARY OF THE INVENTION

This invention provides previously unknown correlations between various polymorphisms and metabolic syndrome phenotypes including metabolic syndrome, obesity, dyslipidemia, high blood pressure (e.g., hypertension), incidence of myocardial infarction, insulin resistance, and/or diabetes. The detection of these polymorphisms, accordingly, provides robust and precise methods and systems for identifying patients that have or are at risk for metabolic syndrome, obesity, dyslipidemia, high blood pressure (e.g., hypertension), incidence of myocardial infarction, insulin resistance, and/or diabetes. In addition, the identification of these polymorphisms provides high-throughput systems and methods for identifying modulators of metabolic syndrome, obesity, dyslipidemia, high blood pressure (e.g., hypertension), incidence of myocardial infarction, insulin resistance, and/or diabetes.

Accordingly, in a first aspect, methods of identifying a metabolic syndrome phenotype, including presence or absence of metabolic syndrome, obesity (e.g., based on BMI or waist circumference), dyslipidemia (e.g., based on HDL or triglyceride levels), high systolic or diastolic blood pressure, hypertension, incidence of myocardial infarction, insulin resistance (e.g., based on HOMA score), diabetes, and/or abnormal insulin levels for an organism or biological sample derived therefrom are provided. The method includes detecting, in the organism or biological sample, a polymorphism of a genetic locus (e.g., a gene or at a locus closely linked thereto). Example genes and other genetic loci are provided in Tables 1-14, 17 and 18 in which the polymorphisms listed are associated with one or more metabolic syndrome phenotypes, including metabolic syndrome, diabetes, incidence of myocardial infarction, hypertension, a high body mass index (BMI), a large waist circumference, a high diastolic blood pressure, a high systolic blood pressure, low HDL (high density lipoprotein) cholesterol levels, high blood triglyceride levels, insulin levels in nondiabetics, insulin levels in non-Mexicans, insulin resistance (based on HOMA score) in nondiabetics, and insulin resistance (based on HOMA score) in non-Mexicans, respectively. Similarly, detecting a polymorphism of Tables 1-14, or a locus closely linked thereto, can be used to identify a polymorphism associated with the metabolic syndrome phenotype. In either case, presence of the relevant polymorphism is correlated to the metabolic syndrome phenotype thereby identifying the relevant phenotype.

Any of the features of metabolic syndrome can constitute the relevant phenotype, e.g., metabolic syndrome, diabetes, incidence of myocardial infarction, hypertension, a high BMI, a large waist circumference, a high diastolic blood pressure, a high systolic blood pressure, low HDL cholesterol levels, high blood triglyceride levels, abnormal insulin levels, insulin resistance, central obesity, dyslipidemia (e.g., atherogenic dyslipidemia), glucose intolerance, a chronic prothrombotic state, a chronic proinflammatory state, etc. The various metabolic syndrome phenotypes overlap with metabolic syndrome, along with the markers used herein to detect them.

The organism or the biological sample can be, or can be derived from, a mammal. For example, the organism can be a human patient, or the biological sample can be derived from a human patient (blood, lymph, skin, tissue, saliva, primary or secondary cell cultures derived therefrom, etc.).

Detecting the polymorphism can include amplifying the polymorphism or a sequence associated therewith and detecting the resulting amplicon. For example, amplifying the polymorphism can include admixing an amplification primer or amplification primer pair with a nucleic acid template isolated from the organism or biological sample. The primer or primer pair is typically complementary or partially complementary to at least a portion of the gene or other polymorphism, or to a proximal sequence thereto, and is capable of initiating nucleic acid polymerization by a polymerase on the nucleic acid template. The amplification can also include extending the primer or primer pair in a DNA polymerization reaction using a polymerase and the template nucleic acid to generate the amplicon. The amplicon can be detected by hybridizing the amplicon to an array, digesting the amplicon with a restriction enzyme, real-time PCR analysis, sequencing of the amplicon, or the like. Optionally, amplification can include performing a polymerase chain reaction (PCR), reverse transcriptase PCR (RT-PCR), or ligase chain reaction (LCR) using nucleic acid isolated from the organism or biological sample as a template in the PCR, RT-PCR, or LCR. Other formats can include allele specific hybridization, single nucleotide extension, or the like.

The polymorphism can be any detectable polymorphism, e.g., a SNP. For example, the allele can be any of those noted in Tables 1-14, 17 and 18. The alleles can positively or negatively correlate to existence of or susceptibility to one or more metabolic syndrome phenotypes, including diabetes incidence of myocardial infarction, high blood pressure, obesity, insulin resistance, and/or dyslipidemia.

Polymorphisms closely linked to the polymorphisms of Tables 1-14, 17 and 18 can be used as markers for metabolic syndrome phenotypes. Such closely linked markers are typically about 20 cM or less, e.g., 15 cM or less, often 10 cM or less and, in certain preferred embodiments, 5 cM or less from the gene or other polymorphism of interest (e.g., an allelic marker locus in Tables 1-14). The linked markers can, of course be closer than 5 cM, e.g., 4, 3, 2, 1, 0.5, 0, 25, 0.1 cM or less from the gene or marker locus of Tables 1-14, 17 or 18. In general, the closer the linkage (or association), the more predictive the linked marker is of an allele of the gene or given marker locus (or association).

In one typical embodiment, correlating the polymorphism is performed by referencing a look up table that comprises correlations between alleles of the polymorphism and the phenotype. This table can be, e.g., a paper or electronic database comprising relevant correlation information. In one aspect, the database can be a multidimensional database comprising multiple correlations and taking multiple correlation relationships into account, simultaneously. Accessing the look up table can include extracting correlation information through a table look-up or can include more complex statistical analysis, such as principle component analysis (PCA), heuristic algorithms that track and/or update correlation information (e.g., neural networks), hidden Markov modeling, or the like.

Correlation information is useful for determining disease susceptibility (e.g., patient susceptibility to metabolic syndrome, obesity, insulin resistance, diabetes, high blood pressure, dyslipidemia, or myocardial infarction), disease diagnosis (e.g., diagnosis of metabolic syndrome), and disease prognosis (e.g., likelihood that conventional therapies such as diet and exercise will be effective, in light of patient genotype). Correlation information may also be useful for determining an appropriate medical treatment regimen for an individual exhibiting or susceptible to one or more metabolic syndrome phenotypes. In addition, for non-human applications, the ability to predict metabolic syndrome, obesity, insulin resistance, diabetes, high blood pressure, dyslipidemia, or myocardial infarction is useful, e.g., to livestock breeders who wish to perform marker-assisted breeding (by conventional or in vitro fertilization (IVF) assisted methods) to control, e.g., fat production in livestock. Thus, where the organism is a non-human mammal, the methods optionally further include selecting the non-human mammal, or germplasm (e.g., sperm or eggs) therefrom, from a population of non-human mammals, based upon the determined correlation to phenotype. The resulting selected non-human mammal can be bred with another non-human mammal (by conventional or IVF assisted methods) to optimize genotype and resulting phenotype in one or more offspring.

Kits that comprise, e.g., probes for identifying the markers herein, e.g., packaged in suitable containers with instructions for correlating detected alleles to a metabolic syndrome phenotype, including presence of or susceptibility to metabolic syndrome, obesity, high blood pressure, diabetes, dyslipidemia, insulin resistance, hypertension, abnormal insulin levels, myocardial infarction, etc. are a feature of the invention as well.

In an additional aspect, methods of identifying modulators of a metabolic syndrome phenotype are provided. The methods include contacting a potential modulator to a gene or gene product, such as a gene listed in Tables 1-14, and/or a gene product corresponding to any of these genes. An effect of the potential modulator on the gene or gene product is detected, thereby identifying whether the potential modulator modulates the metabolic syndrome phenotype. All of the features described above for the alleles, genes, markers, etc., are applicable to these methods as well.

Effects of interest for which one may screen include: (a) increased or decreased expression of any gene product of Tables 1-14, 17 and 18 in the presence of the modulator; (b) a change in the timing or location of expression of any gene product in Tables 1-14, 17 and 18 in the presence of the modulator; (c) a change in localization of proteins encoded by the genes of Tables 1-14, 17 and 18 in the presence of the modulator.

The invention also includes kits for treatment of a metabolic syndrome phenotype. In one aspect, the kit comprises a modulator identified by the method above and instructions for administering the compound to a patient to treat the metabolic syndrome phenotype.

In an additional aspect, systems for identifying a metabolic syndrome phenotype for an organism or biological sample derived therefrom are provided. Such systems include, e.g., a set of marker probes or primers configured to detect at least one allele of one or more gene, polymorphism or linked locus associated with the metabolic syndrome phenotype, wherein the gene or polymorphism comprises or encodes any gene, polymorphism, or gene product of Tables 1-14, 17 and 18. Typically, the set of marker probes or primers can include or detect a nucleotide sequence of Tables 1-14, 17 and 18 or an allele closely linked thereto. The system typically also includes a detector that is configured to detect one or more signal outputs (e.g., light emissions) from the set of marker probes or primers, or an amplicon produced from the set of marker probes or primers, thereby identifying the presence or absence of the allele. System instructions that correlate the presence or absence of the allele with the predicted metabolic syndrome phenotype, thereby identifying the metabolic syndrome phenotype for the organism or biological sample derived therefrom are also a feature of the system. The instructions can include at least one look-up table that includes a correlation between the presence or absence of the one or more alleles and the metabolic syndrome phenotype. The system can further include a sample, which is typically derived from a mammal, including e.g., a genomic DNA, an amplified genomic DNA, a cDNA, an amplified cDNA, RNA, or an amplified RNA.

In one aspect, the invention specifically relates to a method of identifying a metabolic syndrome phenotype for an organism, the method comprising:

detecting, in a biological sample from the organism, a polymorphism of a gene or a locus closely linked thereto, the gene selected from those listed in Tables 1-14, 17 and 18, wherein the polymorphism is associated with the metabolic syndrome phenotype; and,

correlating the polymorphism to the metabolic syndrome phenotype, thereby identifying the metabolic syndrome phenotype.

In one embodiment, the metabolic syndrome phenotype is altered triglyceride levels, where the polymorphism may, for example, be in the MLXIPL region on chromosome 7 at 7q11.23, or can be in or proximal to a gene located in the MLXIPL region on chromosome 7 at 7q11.23, listed in Table 18.

In another embodiment, the metabolic syndrome phenotype is higher plasma triglyceride levels. In this embodiment, the polymorphism may, for example, be a single nucleotide polymorphism (SNP) in the MLXIPL gene or a gene or a locus closely linked thereto. In particular embodiments, the polymorphism is an allele at a SNP selected from the group consisting of: a cytosine at position rs1375388, an adenine at rs1448972, a guanine at rs 6844155, a cytosine at rs4960288, an adenine at rs 12056034, a thymine at rs 17145732, a cytosine at rs 3812316, an adenine at rs799160, a thymidine at rs325, an adenine at rd326, a cytosine at rs328, a cytosine at rs17410914, a thymidine at rs4406409, a cytosine at rs1558861, a guanine at rs2075292, an adenine at rs7124741, an adenine at rs17120139, a cytosine at rs9508032, a cytosine at rs9513115, a cytosine at rs9895521, a cytosine at rs747398, an adenine at rs4824743. Alternatively, or in addition, the polymorphism may be wherein the polymorphism is in the vicinity of the APOA1-APOA3-APOA4-APOA5 cluster. Thus, the polymorphism may be at or proximal to a gene listed in Table 18. In a particular embodiment, the polymorphism is a single nucleotide polymorphism (SNP) in the LPL gene or a gene or a locus closely linked thereto.

In a further embodiment, the metabolic syndrome phenotype is lower high density lipoprotein levels. In this embodiment, the polymorphism may, for example, be an ellele at a single nucleotide polymorphism (SNP) selected from the group consisting of: a thymidine at rs2992753, an adenine at rs2819770, a thymine at rs 17145732, a thymine at rs325, an adenine at rs326, a cytosine at rs328, a thymine at rs9282541, a guanine at rs11858164, a thymine at rs2217332, a guanine at rs711752, a guanine at rs7205804, a cytosine at rs5880, an adenine at rs5882, an adenine at rs1800777, and an adenine at rs4824743.

In a still further embodiment, the metabolic syndrome phenotype is altered blood pressure, such as high blood pressure. In this embodiment, the polymorphism may, for example, be a guanine at rs5174.

In yet another embodiment, the metabolic syndrome phenotype is susceptibility to metabolic syndrome, when the polymorphism may, for example, be a guanine at rs1354746 or a guanine at rs7205804.

In another aspect, the invention concerns a method of identifying a modulator of a metabolic syndrome phenotype, the method comprising administering a modulator of a gene or gene product, wherein the gene or gene product is encoded by a gene or locus listed in Tables 1-14, 17 or 18 to a non-human mammal or ex vivo mammalian cell, and measuring an effect indicative of a metabolic syndrome phenotype.

In a further aspect, the invention concerns method of identifying a metabolic syndrome phenotype for an organism, the method comprising:

detecting, in a biological sample from the organism, a haplotype in a genomic region comprising an allele selected from the alleles listed in Tables 1-14, 17 or 18; and

correlating the haplotype to the metabolic syndrome phenotype, thereby identifying the metabolic syndrome phenotype.

In a still further aspect, the invention concerns a method for identifying a human subject at increased risk for a metabolic syndrome phenotype, comprising using an in vitro assay to detect the presence of a risk allele provided in Table 18 in a human subject that is more frequently present in a population of humans with the metabolic syndrome phenotype than in a population of humans that do not have the metabolic syndrome phenotype, wherein the presence of the risk allele indicates that the human subject has an increased risk for the metabolic syndrome phenotype.

In another aspect, the invention concerns a method for identifying a human subject at increased risk for coronary heart disease, comprising using an in vitro assay to detect the presence of a polymorphism with a linkage disequilibrium of at least r²=0.8 with a risk allele provided in Table 18 in a human subject, wherein the presence of the polymorphism indicates that the human subject has an increased risk for coronary heart disease.

In yet another aspect, the invention concerns a method for determining whether a human subject is at increased risk for a metabolic syndrome phenotype, comprising using an in vitro assay to detect the presence of a haplotype comprising a risk allele provided in Table 18 in a human subject, wherein the presence of the haplotype indicates that the human subject has an increased risk for the metabolic syndrome phenotype.

In an additional aspect, the invention concerns a method for identifying a human subject at increased risk for a metabolic syndrome phenotype, comprising using an in vitro assay to detect the genotype of a SNP provided in Table 18, wherein the genotype of the SNP indicates that the human subject has an increased risk for the metabolic syndrome phenotype.

The invention further provides a kit comprising one or more components for detecting the presence of a risk allele provided in Table 18 in a human subject.

It will be appreciated that the methods, systems and kits above can all be used together in various combinations and that features of the methods can be reflected in the systems and kits, and vice-versa.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fees.

FIG. 1. Principal components analysis of Phase II data. Results are shown for samples projected onto the top two principal components, colored by reported ancestry. The scaling of the two axes is essentially arbitrary. The shape of the Mexican cluster is consistent with PC1 quantifying a sample's European ancestry in this admixed population.

FIG. 2. Genomic context of the MLXIPL (earlier known as WBSCR14) region on chromosome 7q11.23. The lower panel shows pairwise linkage disequilibrium between SNPs with MAF≧0.05 in the Phase II HapMap (release 21a) CEU panel, using Haploview's standard color scheme. The four tested SNPs all have D′>0.9 with the most-associated SNP, rs12316, in the stage 2 data. Association test scores are shown as −log₁₀(P), where P is from Table 18.

### DETAILED DESCRIPTION OF THE INVENTION

The present invention provides correlations between polymorphisms in or proximal to the genes listed in Tables 1-14 and one or more metabolic syndrome phenotypes. Thus, detection of particular polymorphisms in these loci or genes, or their encoded gene products, provides methods for identifying patients that have or are at risk for one or more metabolic syndrome phenotypes, including presence of or predisposition to metabolic syndrome, obesity, insulin resistance, high blood pressure, diabetes, myocardial infarction, and dyslipidemia. Systems for detecting and correlating alleles to metabolic syndrome phenotypes, e.g., for practicing the methods, are also a feature of the invention. In addition, the identification of these polymorphisms provides high-throughput systems and methods for identifying modulators of metabolic syndrome phenotypes.

The following definitions are provided to more clearly identify aspects of the present invention. They should not be imputed to any other related or unrelated application or patent.

### DEFINITIONS

It is to be understood that this invention is not limited to particular embodiments, which can, of course, vary. It is also to be understood that the terminology used herein is for the purpose of describing particular embodiments only, and is not intended to be limiting. As used in this specification and the appended claims, terms in the singular and the singular forms “a,” “an” and “the,” for example, optionally include plural referents unless the content clearly dictates otherwise. Thus, for example, reference to “a probe” optionally includes a plurality of probe molecules; similarly, depending on the context, use of the term “a nucleic acid” optionally includes, as a practical matter, many copies of that nucleic acid molecule. Letter designations for genes or proteins can refer to the gene form and/or the protein form, depending on context. One of skill is fully able to relate the nucleic acid and amino acid forms of the relevant biological molecules by reference to the sequences herein, known sequences and the genetic code.

In the following description, various phases of a case study are referred to as “Phase” or “stage,” which terms are used interchangeably.

Unless otherwise indicated, nucleic acids are written left to right in a 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used in accordance with the definitions set out below.

A “phenotype” is a trait or collection of traits that is/are observable in an individual or population. The trait can be quantitative (a quantitative trait, or QTL) or qualitative.

A “metabolic syndrome phenotype” is a phenotype that displays a predisposition towards developing metabolic syndrome in an individual (e.g., a risk factor for metabolic syndrome), or that displays metabolic syndrome in the individual, or that is a phenotype clinically associated with metabolic syndrome (e.g., obesity, dyslipidemia, and high blood pressure). A phenotype that displays a predisposition for metabolic syndrome, can for example, show a higher likelihood that the syndrome will develop in an individual with the phenotype than in members of the general population under a given set of environmental conditions, such as a high calorie, e.g., high-fat, and/or high-carbohydrate diet, and/or a low physical activity regime. Metabolic syndrome can be characterized by any of a number of metabolic disorders or risk factors, generally considered to most typify metabolic syndrome when more than one of these factors are present in a single individual. The factors include: obesity (in particular, central obesity, which is characterized by disproportionate fat tissue in and around the abdomen); high BMI (body mass index); large waist circumference; hypertension; atherogenic dyslipidemia (these include a family of blood fat disorders including, e.g., high triglycerides and low HDL cholesterol, that can foster plaque buildups in the vascular system, including artery walls); high blood pressure (e.g., 130/85 mm Hg or higher); diabetes, abnormal insulin levels, insulin resistance or glucose intolerance (the body can't properly use insulin or blood sugar); susceptibility to myocardial infarction; a chronic prothrombotic state (e.g., characterized by high fibrinogen or plasminogen activator inhibitor [−1] levels in the blood); and a chronic proinflammatory state (e.g., characterized by higher than normal levels of high-sensitivity C-reactive protein in the blood).

Insulin resistance occurs when the body doesn't respond as well to the insulin that the pancreas is making and glucose is less able to enter the cells. People with insulin resistance may or may not go on to develop type 2 diabetes. Any of a variety of tests in current use can be used to determine insulin resistance, including: the Oral Glucose Tolerance Test (OGTT), Fasting Blood Glucose (FBG), Normal Glucose Tolerance (NGT), Impaired Glucose Tolerance (IGT), Impaired Fasting Glucose (IFG), Homeostasis Model Assessment (HOMA), the Quantitative Insulin Sensitivity Check Index (QUICKI) and the Intravenous Insulin Tolerance Test (IVITT). See also, www.retroconference.org/2002/Posters/12814.pdf; De Vegt (1998) “The 1997 American Diabetes Association criteria versus the 1985 World Health Organization criteria for the diagnosis of abnormal glucose tolerance: poor agreement in the Hoorn Study.” Diab Care 1998, 21:1686-1690; Matthews (1985) “Homeostasis model assessment: insulin resistance and B-cell function from fasting plasma glucose and insulin concentrations in man.” Diabetologia 28:412-419; Katz, A (2000) “Quantitative Insulin Sensitivity Check Index: A Simple, Accurate Method for Assessing Insulin Sensitivity In Humans.” JCE & M 85:2402-2410.

A “polymorphism” is a locus that is variable; that is, within a population, the nucleotide sequence at a polymorphism has more than one version or allele. The term “allele” refers to one of two or more different nucleotide sequences that occur or are encoded at a specific locus, or two or more different polypeptide sequences encoded by such a locus. For example, a first allele can occur on one chromosome, while a second allele occurs on a second homologous chromosome, e.g., as occurs for different chromosomes of a heterozygous individual, or between different homozygous or heterozygous individuals in a population. One example of a polymorphism is a “single nucleotide polymorphism” (SNP), which is a polymorphism at a single nucleotide position in a genome (the nucleotide at the specified position varies between individuals or populations).

An allele “positively” correlates with a trait when it is linked to it and when presence of the allele is an indictor that the trait or trait form will occur in an individual comprising the allele. An allele negatively correlates with a trait when it is linked to it and when presence of the allele is an indicator that a trait or trait form will not occur in an individual comprising the allele.

A marker polymorphism or allele is “correlated” with a specified phenotype (metabolic syndrome, obesity predisposition, insulin resistance, etc.) when it can be statistically linked (positively or negatively) to the phenotype. This correlation is often inferred as being causal in nature, but it need not be simple genetic linkage to (association with) a locus for a trait that underlies the phenotype is sufficient. Such an allele may be “protective” and reduce an individual's likelihood of developing a metabolic syndrome phenotype, or may increase an individual's susceptibility or predisposition to developing a metabolic syndrome phenotype.

A “favorable allele” is an allele at a particular locus that positively correlates with a desirable phenotype, e.g., resistance to obesity, or resistance to metabolic syndrome, or that negatively correlates with an undesirable phenotype, e.g., an allele that negatively correlates with obesity predisposition or predisposition to metabolic syndrome. The desired phenotype can, of course, vary, e.g., in some animal breeding contexts, predisposition to obesity can be desirable, instead of undesirable, as it is in many human populations. A favorable allele of a linked marker is a marker allele that segregates with the favorable allele. A favorable allelic form of a chromosome segment is a chromosome segment that includes a nucleotide sequence that positively correlates with the desired phenotype, or that negatively correlates with the unfavorable phenotype at one or more genetic loci physically located on the chromosome segment.

An “unfavorable allele” is an allele at a particular locus that negatively correlates with a desirable phenotype, or that correlates positively with an undesirable phenotype, e.g., positive correlation to obesity predisposition, or metabolic syndrome predisposition, or negative correlation with obesity resistance or resistance to metabolic syndrome. Here again, the desired phenotype can, of course, vary, e.g., in some animal breeding contexts, predisposition to obesity can be desirable, instead of undesirable, as it is in many human populations. An unfavorable allele of a linked marker is a marker allele that segregates with the unfavorable allele. An unfavorable allelic form of a chromosome segment is a chromosome segment that includes a nucleotide sequence that negatively correlates with the desired phenotype, or positively correlates with the undesirable phenotype at one or more genetic loci physically located on the chromosome segment.

“Allele frequency” refers to the frequency (proportion or percentage) at which an allele is present at a locus within an individual, within a line, or within a population of lines. For example, for an allele “A,” diploid individuals of genotype “AA,” “Aa,” or “aa” have allele frequencies of 1.0, 0.5, or 0.0, respectively. One can estimate the allele frequency within a line or population by averaging the allele frequencies of a sample of individuals from that line or population. Similarly, one can calculate the allele frequency within a population of lines by averaging the allele frequencies of lines that make up the population.

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

A “locus” is a chromosomal position or region. For example, a polymorphic locus is a position or region where a polymorphic nucleic acid, trait determinant, gene or marker is located. In a further example, a “gene locus” is a specific chromosome location in the genome of a species where a specific gene can be found. Similarly, the term “quantitative trait locus” or “QTL” refers to a locus with at least two alleles that differentially affect the expression or alter the variation of a quantitative or continuous phenotypic trait in at least one genetic background, e.g., in at least one breeding population or progeny.

A “marker,” “molecular marker” or “marker nucleic acid” refers to a nucleotide sequence or encoded product thereof (e.g., a protein) used as a point of reference when identifying a locus or a linked locus. A marker can be derived from genomic nucleotide sequence or from expressed nucleotide sequences (e.g., from an RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Nucleic acids are “complementary” when they specifically hybridize in solution, e.g., according to Watson-Crick base pairing rules. A “marker locus” is a locus that can be used to track the presence of a second linked locus, e.g., a linked or correlated locus that encodes or contributes to the population variation of a phenotypic trait. For example, a marker locus can be used to monitor segregation of alleles at a locus, such as a QTL, that are genetically or physically linked to the marker locus. Thus, a “marker allele,” or, alternatively, an “allele of a marker locus” is one of a plurality of polymorphic nucleotide sequences found at a marker locus in a population that is polymorphic for the marker locus. In one aspect, the present invention provides marker loci correlating with a phenotype of interest, e.g., one or more metabolic syndrome phenotypes. Each of the identified markers is expected to be in close or overlapping physical and genetic proximity (resulting in physical and/or genetic linkage) to a genetic element, e.g., a QTL, that contributes to the relevant phenotype. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of allele specific hybridization (ASH), detection of single nucleotide extension, detection of amplified variable sequences of the genome, detection of self-sustained sequence replication, detection of simple sequence repeats (SSRs), detection of single nucleotide polymorphisms (SNPs), or detection of amplified fragment length polymorphisms (AFLPs).

A “genetic map” is a description of genetic linkage (or association) relationships among loci on one or more chromosomes (or linkage groups) within a given species, generally depicted in a diagrammatic or tabular form. “Mapping” is the process of defining the linkage relationships of loci through the use of genetic markers, populations segregating for the markers, and standard genetic principles of recombination frequency. A “map location” is an assigned location on a genetic map relative to linked genetic markers where a specified marker can be found within a given species. The term “chromosome segment” or designates a contiguous linear span of genomic DNA that resides on a single chromosome. Similarly, a “haplotype” is a set of genetic loci found in the heritable material of an individual or population (the set can be a contiguous or non-contiguous). In the context of the present invention genetic elements such as one or more alleles herein and one or more linked marker alleles can be located within a chromosome segment and are also, accordingly, genetically linked, a specified genetic recombination distance of less than or equal to 20 centimorgan (cM) or less, e.g., 15 cM or less, often 10 cM or less, e.g., about 9, 8, 7, 6, 5, 4, 3, 2, 1, 0.75, 0.5, 0.25, or 0.1 CM or less. That is, two closely linked genetic elements within a single chromosome segment undergo recombination during meiosis with each other at a frequency of less than or equal to about 20%, e.g., about 19%, 18%, 17%, 16%, 15%, 14%, 13%, 12%, 11%, 10%, 9%, 8%, 7%, 6%, 5%, 4%, 3%, 2%, 1%, 0.75%, 0.5%, 0.25%, or 0.1% or less.

A “genetic recombination frequency” is the frequency of a recombination event between two genetic loci. Recombination frequency can be observed by following the segregation of markers and/or traits during meiosis. In the context of this invention, a marker locus is “associated with” another marker locus or some other locus (for example, an obesity or metabolic syndrome locus), when the relevant loci are part of the same linkage group due to association and are in linkage disequilibrium. This occurs when the marker locus and a linked locus are found together in progeny more frequently than if the loci segregate randomly. Similarly, a marker locus can also be associated with a trait, e.g., a marker locus can be “associated with” a given trait when the marker locus is in linkage disequilibrium with the trait. The term “linkage disequilibrium” refers to a non-random segregation of genetic loci or traits (or both). In either case, linkage disequilibrium implies that the relevant loci are within sufficient physical proximity along a length of a chromosome so that they segregate together with greater than random frequency (in the case of co-segregating traits, the loci that underlie the traits are in sufficient proximity to each other). Linked loci co-segregate more than 50% of the time, e.g., from about 51% to about 100% of the time. Advantageously, the two loci are located in close proximity such that recombination between homologous chromosome pairs does not occur between the two loci during meiosis with high frequency, e.g., such that closely linked loci co-segregate at least about 80% of the time, more preferably at least about 85% of the time, still more preferably at least 90% of the time, e.g., 91%, 92%, 93%, 94%, 95%, 96%, 97%, 98%, 99%, 99.5%, 99.75%, or 99.90% or more of the time.

In some embodiments, a polymorphism with a linkage disequilibrium of at least r²=0.89, 0.90, 0.91, 0.92, 0.93, 0.94, 0.95, 0.96, 0.97, 0.98 or 0.99 with a polymorphism associated with a metabolic syndrome phenotype (such as those provided in the tables herein) in a human subject is detected. Techniques for determining whether a polymorphism is in linkage disequilibrium with a risk allele are well-known to the one of skill in the art. See, e.g., the following for information concerning linkage disequilibrium in the human genome: Wall et al., “Haplotype blocks and linkage disequilibrium in the human genome”, Nat Rev Genet. 2003 August; 4(8):587-97; Garner et al., “On selecting markers for association studies: patterns of linkage disequilibrium between two and three diallelic loci”, Genet Epidemiol. 2003 January; 24(1):57-67; Ardlie et al., “Patterns of linkage disequilibrium in the human genome”, Nat Rev Genet. 2002 April; 3(4):299-309 (erratum in Nat Rev Genet 2002 July; 3(7):566); and Remm et al., “High-density genotyping and linkage disequilibrium in the human genome using chromosome 22 as a model”; Curr Opin Chem Biol. 2002 February; 6(1):24-30; Haldane J B S (1919) The combination of linkage values, and the calculation of distances between the loci of linked factors. J Genet 8:299-309; Mendel, G. (1866) Versuche uber Pflanzen-Hybriden. Verhandlungen des naturforschenden Vereines in Brunn [Proceedings of the Natural History Society of Brunn]; Lewin B (1990) Genes IV Oxford University Press, New York, USA; Hartl D L and Clark A G (1989) Principles of Population Genetics 2.sup.nd ed. Sinauer Associates, Inc. Sunderland, Mass., USA; Gillespie J H (2004) Population Genetics: A Concise Guide. 2.sup.nd ed. Johns Hopkins University Press. USA; Lewontin R C (1964) The interaction of selection and linkage. I. General considerations; heterotic models. Genetics 49:49-67; Hoel P G (1954) Introduction to Mathematical Statistics 2.sup.nd ed. John Wiley & Sons, Inc. New York, USA; Hudson R R (2001) Two-locus sampling distributions and their application. Genetics 159:1805-1817; Dempster A P, Laird N M, Rubin D B (1977) Maximum likelihood from incomplete data via the EM algorithm. J R Stat Soc 39:1-38; Excoffier L, Slatkin M (1995) Maximum-likelihood estimation of molecular haplotype frequencies in a diploid population. Mol Biol Evol 12(5):921-927; Tregouet D A, Escolano S, Tiret L, Mallet A, Golmard J L (2004) A new algorithm for haplotype-based association analysis: the Stochastic-EM algorithm. Ann Hum Genet 68(Pt 2):165-177; Long A D and Langley C H (1999) The power of association studies to detect the contribution of candidate genetic loci to variation in complex traits. Genome Research 9:720-731; Agresti A (1990) Categorical Data Analysis. John Wiley & Sons, Inc. New York, USA; Lange K (1997) Mathematical and Statistical Methods for Genetic Analysis. Springer-Verlag New York, Inc. New York, USA; The International HapMap Consortium (2003) The International HapMap Project. Nature 426:789-796; The International HapMap Consortium (2005) A haplotype map of the human genome. Nature 437:1299-1320; Thorisson G A, Smith A V, Krishnan L, Stein L D (2005), The International HapMap Project Web Site. Genome Research 15:1591-1593; McVean G, Spencer C C A, Chaix R (2005) Perspectives on human genetic variation from the HapMap project. PLoS Genetics 1(4):413-418; Hirschhorn J N, Daly M J (2005) Genome-wide association studies for common diseases and complex traits. Nat Genet 6:95-108; Schrodi S J (2005) A probabilistic approach to large-scale association scans: a semi-Bayesian method to detect disease-predisposing alleles. SAGMB 4(1):3 1; Wang W Y S, Barratt B J, Clayton D G, Todd J A (2005) Genome-wide association studies: theoretical and practical concerns. Nat Rev Genet 6:109-118. Pritchard J K, Przeworski M (2001) Linkage disequilibrium in humans: models and data. Am J Hum Genet 69:1-14. The parameter r² is commonly used in the genetics art to characterize the extent of linkage disqulibirium between two genetic loci (see, e.g., Hudson et al. (2001) Genetics 159:1805-1817).

The phrase “closely linked,” in the present application, means that recombination between two linked loci (e.g., a SNP such as one identified in Tables 1-14 herein and a second linked allele) occurs with a frequency of equal to or less than about 20%. Put another way, the closely (or “tightly”) linked loci co-segregate at least 80% of the time. Marker loci are especially useful in the present invention when they are closely linked to target loci (e.g., QTL for metabolic syndrome, obesity predisposition, and/or insulin resistance, or, alternatively, simply other marker loci). The more closely a marker is linked to a target locus, the better an indicator for the target locus that the marker is. Thus, in one embodiment, tightly linked loci such as a marker locus and a second locus display an inter-locus recombination frequency of about 20% or less, e.g., 15% or less, e.g., 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci (e.g., a marker locus and a target locus such as a QTL) display a recombination frequency of about 1% or less, e.g., about 0.75% or less, more preferably about 0.5% or less, or yet more preferably about 0.25% or less, or still more preferably about 0.1% or less. Two loci that are localized to the same chromosome, and at such a distance that recombination between the two loci occurs at a frequency of less than about 20%, e.g., 15%, more preferably 10% (e.g., about 9%, 8%, 7%, 6%, 5%, 4%, 3%, 2%, 1%, 0.75%, 0.5%, 0.25%, 0.1% or less) are also said to be “proximal to” each other. When referring to the relationship between two linked genetic elements, such as a genetic element contributing to a trait and a proximal marker, “coupling” phase linkage indicates the state where the “favorable” allele at the trait locus is physically associated on the same chromosome strand as the “favorable” allele of the respective linked marker locus. In coupling phase, both favorable alleles are inherited together by progeny that inherit that chromosome strand. In “repulsion” phase linkage, the “favorable” allele at the locus of interest (e.g., a QTL for obesity or metabolic syndrome) is physically associated on the same chromosome strand as an “unfavorable” allele at the proximal marker locus, and the two “favorable” alleles are not inherited together (i.e., the two loci are “out of phase” with each other).

The term “amplifying” in the context of nucleic acid amplification is any process whereby additional copies of a selected nucleic acid (or a transcribed form thereof) are produced. Typical amplification methods include various polymerase based replication methods, including the polymerase chain reaction (PCR), ligase mediated methods such as the ligase chain reaction (LCR) and RNA polymerase based amplification (e.g., by transcription) methods. An “amplicon” is an amplified nucleic acid, e.g., a nucleic acid that is produced by amplifying a template nucleic acid by any available amplification method (e.g., PCR, LCR, transcription, or the like).

A “genomic nucleic acid” is a nucleic acid that corresponds in sequence to a heritable nucleic acid in a cell. Common examples include nuclear genomic DNA and amplicons thereof. A genomic nucleic acid is, in some cases, different from a spliced RNA, or a corresponding cDNA, in that the spliced RNA or cDNA is processed, e.g., by the splicing machinery, to remove introns. Genomic nucleic acids optionally comprise non-transcribed (e.g., chromosome structural sequences, promoter regions, enhancer regions, etc.) and/or non-translated sequences (e.g., introns), whereas spliced RNA/cDNA typically do not have non-transcribed sequences or introns. A “template genomic nucleic acid” is a genomic nucleic acid that serves as a template in an amplification reaction (e.g., a polymerase based amplification reaction such as PCR, a ligase mediated amplification reaction such as LCR, a transcription reaction, or the like).

An “exogenous nucleic acid” is a nucleic acid that is not native to a specified system (e.g., a germplasm, cell, individual, etc.), with respect to sequence, genomic position, or both. As used herein, the terms “exogenous” or “heterologous” as applied to polynucleotides or polypeptides typically refers to molecules that have been artificially supplied to a biological system (e.g., a cell, an individual, etc.) and are not native to that particular biological system. The terms can indicate that the relevant material originated from a source other than a naturally occurring source, or can refer to molecules having a non-natural configuration, genetic location or arrangement of parts.

The term “introduced” when referring to translocating a heterologous or exogenous nucleic acid into a cell refers to the incorporation of the nucleic acid into the cell using any methodology. The term encompasses such nucleic acid introduction methods as “transfection,” “transformation” and “transduction.”

As used herein, the term “vector” is used in reference to polynucleotides or other molecules that transfer nucleic acid segment(s) into a cell. The term “vehicle” is sometimes used interchangeably with “vector.” A vector optionally comprises parts which mediate vector maintenance and enable its intended use (e.g., sequences necessary for replication, genes imparting drug or antibiotic resistance, a multiple cloning site, operably linked promoter/enhancer elements which enable the expression of a cloned gene, etc.). Vectors are often derived from plasmids, bacteriophages, or plant or animal viruses. A “cloning vector” or “shuttle vector” or “subcloning vector” contains operably linked parts that facilitate subcloning steps (e.g., a multiple cloning site containing multiple restriction endonuclease sites).

The term “expression vector” as used herein refers to a vector comprising operably linked polynucleotide sequences that facilitate expression of a coding sequence in a particular host organism (e.g., a bacterial expression vector or a mammalian cell expression vector). Polynucleotide sequences that facilitate expression in prokaryotes typically include, e.g., a promoter, an operator (optional), and a ribosome binding site, often along with other sequences. Eukaryotic cells can use promoters, enhancers, termination and polyadenylation signals and other sequences that are generally different from those used by prokaryotes.

A specified nucleic acid is “derived from” a given nucleic acid when it is constructed using the given nucleic acid's sequence, or when the specified nucleic acid is constructed using the given nucleic acid.

A “gene” is one or more sequence(s) of nucleotides in a genome that together encode one or more expressed molecule, e.g., an RNA, or polypeptide. The gene can include coding sequences that are transcribed into RNA which may then be translated into a polypeptide sequence, and can include associated structural or regulatory sequences that aid in replication or expression of the gene. Genes of interest in the present invention include any gene or gene product listed in Tables 1-14.

A “genotype” is the genetic constitution of an individual (or group of individuals) at one or more genetic loci. Genotype is defined by the allele(s) of one or more known loci of the individual, typically, the compilation of alleles inherited from its parents. A “haplotype” is the genotype of an individual at a plurality of genetic loci on a single DNA strand. Typically, the genetic loci described by a haplotype are physically and genetically linked, i.e., on the same chromosome strand.

A “set” of markers or probes refers to a collection or group of markers or probes, or the data derived therefrom, used for a common purpose, e.g., identifying an individual with a specified phenotype (e.g., a metabolic syndrome phenotype, including metabolic syndrome, insulin resistance, obesity, high blood pressure, dyslipidemia, diabetes, myocardial infarction, etc.). Frequently, data corresponding to the markers or probes, or derived from their use, is stored in an electronic medium. While each of the members of a set possess utility with respect to the specified purpose, individual markers selected from the set as well as subsets including some, but not all of the markers, are also effective in achieving the specified purpose.

A “look up table” is a table that correlates one form of data to another, or one or more forms of data with a predicted outcome to which the data is relevant. For example, a look up table can include a correlation between allele data and a predicted trait that an individual comprising one or more given alleles is likely to display. These tables can be, and typically are, multidimensional, e.g., taking multiple alleles into account simultaneously, and, optionally, taking other factors into account as well, such as genetic background, e.g., in making a trait prediction.

A “computer readable medium” is an information storage media that can be accessed by a computer using an available or custom interface. Examples include memory (e.g., ROM or RAM, flash memory, etc.), optical storage media (e.g., CD-ROM), magnetic storage media (computer hard drives, floppy disks, etc.), punch cards, and many others that are commercially available. Information can be transmitted between a system of interest and the computer, or to or from the computer or to or from the computer readable medium for storage or access of stored information. This transmission can be an electrical transmission, or can be made by other available methods, such as an IR link, a wireless connection, or the like.

“System instructions” are instruction sets that can be partially or fully executed by the system. Typically, the instruction sets are present as system software.

A “translation product” is a product (typically a polypeptide) produced as a result of the translation of a nucleic acid. A “transcription product” is a product (e.g., an RNA, such as an mRNA, a catalytic or biologically active RNA, or the like) produced as a result of transcription of a nucleic acid (e.g., a DNA).

An “array” is an assemblage of elements. The assemblage can be spatially ordered (a “patterned array”) or disordered (a “randomly patterned” array). The array can form or comprise one or more functional elements (e.g., a probe region on a microarray) or it can be non-functional.

As used herein, the term “SNP” or “single nucleotide polymorphism” refers to a genetic variation between individuals; e.g., a single nitrogenous base position in the DNA of organisms that is variable. As used herein, “SNPs” is the plural of SNP. Of course, when one refers to DNA herein, such reference may include derivatives of the DNA such as amplicons, RNA transcripts thereof, etc.

The term “genomic inflation factor” or “inflation factor” refers to a comparison of unassociated genetic markers with those of control subjects for potential differences in allele frequency related to imperfect matching between case subjects and control.

The term “nonsynonymous SNP” is used to refer to a SNP that leads to a change in the amino acid sequence of the gene's resulting protein and that may therefore affect the three-dimensional structure and its function.

Overview

The invention includes new correlations between the genes, products or loci of Tables 1-14, 17 and 18 and a variety of metabolic syndrome phenotypes, including presence of or predisposition to metabolic syndrome, obesity, insulin resistance, high blood pressure, dyslipidemia, diabetes, and myocardial infarction. Certain alleles in, and linked to, these genes or gene products are predictive of the likelihood that an individual possessing the relevant alleles will develop one or more of these disorders. Accordingly, detection of these alleles, by any available method, can be used for diagnostic purposes such as early detection of susceptibility to a metabolic syndrome phenotype, prognosis for patients that present with the metabolic syndrome phenotype, and in assisting diagnosis, e.g., where current criteria are insufficient for a definitive diagnosis. In addition, because fat production in livestock is important to livestock breeders, it is possible to perform marker assisted selection (MAS) on livestock and livestock germplasm using such allele correlations to select for or against metabolic syndrome phenotypes, e.g., obesity.

The identification that the genes or gene products of Tables 1-14, 17 and 18 are correlated to the metabolic syndrome phenotypes noted above also provides a platform for screening potential modulators of metabolic syndrome phenotypes. Modulators of the activity of any of these genes or their encoded proteins are expected to have an effect on metabolic syndrome, obesity, insulin resistance, high blood pressure, dyslipidemia, diabetes, and risk of myocardial infarction. Thus, methods of screening, systems for screening and the like, are features of the invention. Modulators identified by these screening approaches are also a feature of the invention.

Kits for the diagnosis and treatment of metabolic syndrome, obesity, insulin resistance, high blood pressure, dyslipidemia, diabetes, and risk of myocardial infarction, e.g., comprising probes to identify relevant alleles, packaging materials, and instructions for correlating detection of relevant alleles to metabolic syndrome phenotypes are also a feature of the invention. These kits can also include modulators of the relevant disease and/or instructions for treating patients using conventional methods.

Methods of Identifying Metabolic Syndrome Phenotypes

As noted, the invention provides the discovery that certain genes or other loci of Tables 1-14 are linked to metabolic syndrome phenotypes. Thus, by detecting markers (e.g., the SNPs in Tables 1-14, 17 and 18 or loci closely linked thereto) that correlate, positively or negatively, with the metabolic syndrome phenotypes, it can be determined whether an individual or population is likely to be susceptible to these phenotypes. This provides enhanced early detection options to identify patients that are likely to eventually suffer from these phenotypes, making it possible, in some cases, to prevent actual development of metabolic syndrome phenotypes by taking early preventative action (e.g., any existing therapy such as diet, exercise, available medications, etc.). In addition, use of the various markers herein also adds certainty to existing diagnostic techniques for identifying whether a patient is suffering from, e.g., metabolic syndrome, which can be somewhat ambiguous using previous methods, e.g., as discussed in the Background of the Invention, above. Furthermore, knowledge of whether there is a molecular basis for metabolic syndrome phenotypes can also assist in determining patient prognosis, e.g., by providing an indication of how likely it is that a patient can respond to conventional therapy for the relevant disorder, or whether more serious options such as gastric surgery are likely to be necessary. Disease treatment can also be targeted based on what type of molecular disorder the patient displays.

In non-human subjects (e.g., non-human mammals such as livestock), it is also possible to use this information both for disease diagnosis and prevention (e.g., treatment of pets such as dogs and cats, etc.) as in humans. In addition, it is possible to perform marker-assisted animal breeding to enhance either fat production or lean meat production, depending on what is desired. In brief, livestock animals or germplasm can be selected for marker alleles that positively or negatively correlate with one or more metabolic syndrome phenotypes without actually raising the livestock and measuring for the desired trait. Marker assisted selection (MAS) is a powerful shortcut to selecting for desired phenotypes and for introgressing desired traits into livestock herds (e.g., introgressing desired traits into elite herd populations). MAS is easily adapted to high throughput molecular analysis methods that can quickly screen genetic material for the markers of interest, and is much more cost effective than raising and observing livestock for visible traits.

Detection methods for detecting relevant alleles can include any available method, e.g., amplification technologies. For example, detection can include amplifying the polymorphism or a sequence associated therewith and detecting the resulting amplicon. This can include admixing an amplification primer or amplification primer pair with a nucleic acid template isolated from the organism or biological sample (e.g., comprising the SNP or other polymorphism), e.g., where the primer or primer pair is complementary or partially complementary to at least a portion of the gene or tightly linked polymorphism, or to a sequence proximal thereto. The primer is typically capable of initiating nucleic acid polymerization by a polymerase on the nucleic acid template. The primer or primer pair is extended, e.g., in a DNA polymerization reaction (PCR, RT-PCR, etc.) comprising a polymerase and the template nucleic acid to generate the amplicon. The amplicon is detected by any available detection process, e.g., sequencing, hybridizing the amplicon to an array (or affixing the amplicon to an array and hybridizing probes to it), digesting the amplicon with a restriction enzyme (e.g., RFLP), real-time PCR analysis, single nucleotide extension, allele-specific hybridization, or the like.

The correlation between a detected polymorphism and a trait can be performed by any method that can identify a relationship between an allele and a phenotype. Most typically, these methods involve referencing a look up table that comprises correlations between alleles of the polymorphism and the phenotype. The table can include data for multiple allele-phenotype relationships and can take account of additive or other higher order effects of multiple allele-phenotype relationships, e.g., through the use of statistical tools such as principle component analysis, heuristic algorithms, etc.

Within the context of these methods, the following discussion first focuses on how markers and alleles are linked and how this phenomenon can be used in the context of methods for diagnosing or prognosticating metabolic syndrome phenotypes and then focuses on marker detection methods. Additional sections below discuss data analysis.

Markers, Linkage and Alleles

In traditional linkage (or association) analysis, no direct knowledge of the physical relationship of genes on a chromosome is required. Mendel's first law is that factors of pairs of characters are segregated, meaning that alleles of a diploid trait separate into two gametes and then into different offspring. Classical linkage analysis can be thought of as a statistical description of the relative frequencies of cosegregation of different traits. Linkage analysis is the well characterized descriptive framework of how traits are grouped together based upon the frequency with which they segregate together. That is, if two non-allelic traits are inherited together with a greater than random frequency, they are said to be “linked.” The frequency with which the traits are inherited together is the primary measure of how tightly the traits are linked, i.e., traits which are inherited together with a higher frequency are more closely linked than traits which are inherited together with lower (but still above random) frequency. Traits are linked because the genes which underlie the traits reside near one another on the same chromosome. The further apart on a chromosome the genes reside, the less likely they are to segregate together, because homologous chromosomes recombine during meiosis. Thus, the further apart on a chromosome the genes reside, the more likely it is that there will be a recombination event during meiosis that will result in two genes segregating separately into progeny.

A common measure of linkage (or association) is the frequency with which traits cosegregate. This can be expressed as a percentage of cosegregation (recombination frequency) or, also commonly, in centiMorgans (cM), which are actually a reciprocal unit of recombination frequency. The cM is named after the pioneering geneticist Thomas Hunt Morgan and is a unit of measure of genetic recombination frequency. One cM is equal to a 1% chance that a trait at one genetic locus will be separated from a trait at another locus due to recombination in a single generation (meaning the traits segregate together 99% of the time). Because chromosomal distance is approximately proportional to the frequency of recombination events between traits, there is an approximate physical distance that correlates with recombination frequency. For example, in humans, 1 cM correlates, on average, to about 1 million base pairs (1 Mbp).

Marker loci are themselves traits and can be assessed according to standard linkage analysis by tracking the marker loci during segregation. Thus, in the context of the present invention, one cM is equal to a 1% chance that a marker locus will be separated from another locus (which can be any other trait, e.g., another marker locus, or another trait locus that encodes a QTL for metabolic syndrome, insulin resistance, obesity, high blood pressure, dyslipidemia, diabetes, myocardial infarction, etc.), due to recombination in a single generation. The markers herein, e.g., those listed in Tables 1-14, can correlate with metabolic syndrome, insulin resistance, obesity, high blood pressure, dyslipidemia, diabetes and/or myocardial infarction. This means that the markers comprise or are sufficiently proximal to a QTL for metabolic syndrome, insulin resistance, obesity, high blood pressure, dyslipidemia, diabetes and/or myocardial infarction that they can be used as a predictor for the trait itself. This is extremely useful in the context of disease diagnosis and, in livestock applications, for marker assisted selection (MAS).

From the foregoing, it is clear that any marker that is linked to a trait locus of interest (e.g., in the present case, a QTL or identified linked marker locus for a metabolic syndrome phenotype, e.g., as in Tables 1-14, 17 or 18) can be used as a marker for that trait. Thus, in addition to the markers noted in Tables 1-14, 17, and 18, other markers closely linked to the markers itemized in Tables 1-14, 17 and 18 can also usefully predict the presence of the marker alleles indicated in Tables 1-14, 17 and 18 (and, thus, the relevant phenotypic trait). Such linked markers are particularly useful when they are sufficiently proximal to a given locus so that they display a low recombination frequency with the given locus. In the present invention, such closely linked markers are a feature of the invention. Closely linked loci display a recombination frequency with a given marker of about 20% or less (the given marker is within 20 cM of the given marker). Put another way, closely linked loci co-segregate at least 80% of the time. More preferably, the recombination frequency is 10% or less, e.g., 9%, 8%, 7%, 6%, 5%, 4%, 3%, 2%, 1%, 0.5%, 0.25%, or 0.1% or less. In one typical class of embodiments, closely linked loci are within 5 cM or less of each other.

As one of skill in the art will recognize, recombination frequencies (and, as a result, map positions) can vary depending on the map used (and the markers that are on the map). Additional markers that are closely linked to (e.g., within about 20 cM, or more preferably within about 10 cM of) the markers identified in Tables 1-14 may readily be used for identification of QTL for metabolic syndrome, insulin resistance, obesity, high blood pressure, dyslipidemia, diabetes and/or myocardial infarction.

Marker loci are especially useful in the present invention when they are closely linked to target loci (e.g., QTL for metabolic syndrome, insulin resistance, obesity, high blood pressure, dyslipidemia, diabetes and/or myocardial infarction, or, alternatively, simply other marker loci, such as those itemized in Tables 1-14, 17 and 18 that are, themselves linked to such QTL) that they are being used as markers for. The more closely a marker is linked to a target locus that encodes or affects a phenotypic trait, the better an indicator for the target locus that the marker is (due to the reduced cross-over frequency between the target locus and the marker). Thus, in one embodiment, closely linked loci such as a marker locus and a second locus (e.g., a given marker locus of Tables 1-14, 17 and 18 and an additional second locus) display an inter-locus cross-over frequency of about 20% or less, e.g., 15% or less, preferably 10% or less, more preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci (e.g., a marker locus and a target locus such as a QTL) display a recombination a frequency of about 1% or less, e.g., about 0.75% or less, more preferably about 0.5% or less, or yet more preferably about 0.25% or 0.1% or less. Thus, the loci are about 20 cM, 19 cM, 18 cM, 17 cM, 16 cM, 15 cM, 14 cM, 13 cM, 12 cM, 11 cM, 10 cM, 9 cM, 8 cM, 7 cM, 6 cM, 5 cM, 4 cM, 3 cM, 2 cM, 1 cM, 0.75 cM, 0.5 cM, 0.25 cM, 0 or 0.1 cM or less apart. Put another way, two loci that are localized to the same chromosome, and at such a distance that recombination between the two loci occurs at a frequency of less than 20% (e.g., about 19%, 18%, 17%, 16%, 15%, 14%, 13%, 12%, 11%, 10%, 9%, 8%, 7%, 6%, 5%, 4%, 3%, 2%, 1%, 0.75%, 0.5%, 0.25%, 0.1% or less) are said to be “proximal to” each other. In one aspect, linked markers are within 100 kb (which correlates in humans to about 0.1 cM, depending on local recombination rate), e.g., 50 kb, or even 20 kb or less of each other.

When referring to the relationship between two genetic elements, such as a genetic element contributing to one or more metabolic syndrome phenotypes and a proximal marker, “coupling” phase linkage indicates the state where the “favorable” allele at the locus is physically associated on the same chromosome strand as the “favorable” allele of the respective linked marker locus. In coupling phase, both favorable alleles are inherited together by progeny that inherit that chromosome strand. In “repulsion” phase linkage, the “favorable” allele at the locus of interest (e.g., a QTL for metabolic syndrome, insulin resistance, obesity, high blood pressure, dyslipidemia, diabetes and/or myocardial infarction) is physically linked with an “unfavorable” allele at the proximal marker locus, and the two “favorable” alleles are not inherited together (i.e., the two loci are “out of phase” with each other).

In addition to tracking SNP and other polymorphisms in the genome, and in corresponding expressed nucleic acids and polypeptides, expression level differences between individuals or populations for the gene products of Tables 1-14, 17 and 18 in either mRNA or protein form, can also correlate to a metabolic syndrome phenotype. Accordingly, markers of the invention can include any of, e.g.: genomic loci, transcribed nucleic acids, spliced nucleic acids, expressed proteins, levels of transcribed nucleic acids, levels of spliced nucleic acids, and levels of expressed proteins.

Marker Amplification Strategies

Amplification primers for amplifying markers (e.g., marker loci) and suitable probes to detect such markers or to genotype a sample with respect to multiple marker alleles, are a feature of the invention. In Tables 1-14, 17 and 18, specific loci for amplification are provided, along with amplicon sequences that one of skill can easily use (optionally in conjunction with known flanking sequences) in the design of such primers. For example, primer selection for long-range PCR is described in U.S. Pat. No. 6,898,531 and U.S. Ser. No. 10/236,480, filed Sep. 5, 2002; for short-range PCR, U.S. Ser. No. 10/341,832, filed Jan. 14, 2003 provides guidance with respect to primer selection. Also, there are publicly available programs such as “Oligo” available for primer design. With such available primer selection and design software, the publicly available human genome sequence and the polymorphism locations as provided in Tables 1-14, 17 and 18, one of skill can design primers to amplify the SNPs of the present invention. Further, it will be appreciated that the precise probe to be used for detection of a nucleic acid comprising a SNP (e.g., an amplicon comprising the SNP) can vary, e.g., any probe that can identify the region of a marker amplicon to be detected can be used in conjunction with the present invention. Further, the configuration of the detection probes can, of course, vary. Thus, the invention is not limited to the sequences recited herein.

Indeed, it will be appreciated that amplification is not a requirement for marker detection—for example, one can directly detect unamplified genomic DNA simply by performing a Southern blot on a sample of genomic DNA. Procedures for performing Southern blotting, standard amplification (PCR, LCR, or the like) and many other nucleic acid detection methods are well established and are taught, e.g., in Sambrook et al., Molecular Cloning—A Laboratory Manual (3rd Ed.), Vol. 1-3, Cold Spring Harbor Laboratory, Cold Spring Harbor, N.Y., 2000 (“Sambrook”); Current Protocols in Molecular Biology, F. M. Ausubel et al., eds., Current Protocols, a joint venture between Greene Publishing Associates, Inc. and John Wiley & Sons, Inc., (supplemented through 2002) (“Ausubel”)) and PCR Protocols A Guide to Methods and Applications (Innis et al. eds) Academic Press Inc. San Diego, Calif. (1990) (Innis).

Separate detection probes can also be omitted in amplification/detection methods, e.g., by performing a real time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization).

Typically, molecular markers are detected by any established method available in the art, including, without limitation, allele specific hybridization (ASH), detection of single nucleotide extension, array hybridization (optionally including ASH), or other methods for detecting single nucleotide polymorphisms (SNPs), amplified fragment length polymorphism (AFLP) detection, amplified variable sequence detection, randomly amplified polymorphic DNA (RAPD) detection, restriction fragment length polymorphism (RFLP) detection, self-sustained sequence replication detection, simple sequence repeat (SSR) detection, single-strand conformation polymorphisms (SSCP) detection, isozyme marker detection, northern analysis (where expression levels are used as markers), quantitative amplification of mRNA or cDNA, or the like. While the exemplary markers provided in the table herein are SNP markers, any of the aforementioned marker types can be employed in the context of the invention to identify linked loci that affect or effect metabolic syndrome, insulin resistance, obesity, high blood pressure, dyslipidemia, diabetes and/or myocardial infarction.

Example Techniques for Marker Detection

The invention provides molecular markers that comprise or are linked to QTL for metabolic syndrome, insulin resistance, obesity, high blood pressure, dyslipidemia, diabetes and/or myocardial infarction. The markers find use in disease predisposition diagnosis, prognosis, treatment and for marker assisted selection for desired traits in livestock. It is not intended that the invention be limited to any particular method for the detection of these markers.

Markers corresponding to genetic polymorphisms between members of a population can be detected by numerous methods well-established in the art (e.g., PCR-based sequence specific amplification, restriction fragment length polymorphisms (RFLPs), isozyme markers, northern analysis, allele specific hybridization (ASH), array based hybridization, amplified variable sequences of the genome, self-sustained sequence replication, simple sequence repeat (SSR), single nucleotide polymorphism (SNP), random amplified polymorphic DNA (“RAPD”) or amplified fragment length polymorphisms (AFLP). In one additional embodiment, the presence or absence of a molecular marker is determined simply through nucleotide sequencing of the polymorphic marker region. Any of these methods are readily adapted to high throughput analysis.

Some techniques for detecting genetic markers utilize hybridization of a probe nucleic acid to nucleic acids corresponding to the genetic marker (e.g., amplified nucleic acids produced using genomic DNA as a template). Hybridization formats, including, but not limited to: solution phase, solid phase, mixed phase, or in situ hybridization assays are useful for allele detection. An extensive guide to the hybridization of nucleic acids is found in Tijssen (1993) Laboratory Techniques in Biochemistry and Molecular Biology—Hybridization with Nucleic Acid Probes Elsevier, New York, as well as in Sambrook, Berger and Ausubel.

For example, markers that comprise restriction fragment length polymorphisms (RFLP) are detected, e.g., by hybridizing a probe which is typically a sub-fragment (or a synthetic oligonucleotide corresponding to a sub-fragment) of the nucleic acid to be detected to restriction digested genomic DNA. The restriction enzyme is selected to provide restriction fragments of at least two alternative (or polymorphic) lengths in different individuals or populations. Determining one or more restriction enzyme that produces informative fragments for each allele of a marker is a simple procedure, well known in the art. After separation by length in an appropriate matrix (e.g., agarose or polyacrylamide) and transfer to a membrane (e.g., nitrocellulose, nylon, etc.), the labeled probe is hybridized under conditions which result in equilibrium binding of the probe to the target followed by removal of excess probe by washing.

Nucleic acid probes to the marker loci can be cloned and/or synthesized. Any suitable label can be used with a probe of the invention. Detectable labels suitable for use with nucleic acid probes include, for example, any composition detectable by spectroscopic, radioisotopic, photochemical, biochemical, immunochemical, electrical, optical or chemical means. Useful labels include biotin for staining with labeled streptavidin conjugate, magnetic beads, fluorescent dyes, radiolabels, enzymes, and calorimetric labels. Other labels include ligands that bind to antibodies labeled with fluorophores, chemiluminescent agents, and enzymes. A probe can also constitute radiolabelled PCR primers that are used to generate a radiolabelled amplicon. Labeling strategies for labeling nucleic acids and corresponding detection strategies can be found, e.g., in Haugland (2003) Handbook of Fluorescent Probes and Research Chemicals Ninth Edition by Molecular Probes, Inc. (Eugene Oreg.). Additional details regarding marker detection strategies are found below.

Amplification-Based Detection Methods

PCR, RT-PCR and LCR are in particularly broad use as amplification and amplification-detection methods for amplifying nucleic acids of interest (e.g., those comprising marker loci), facilitating detection of the nucleic acids of interest. Details regarding the use of these and other amplification methods can be found in any of a variety of standard texts, including, e.g., Sambrook, Ausubel, and Berger. Many available biology texts also have extended discussions regarding PCR and related amplification methods. One of skill will appreciate that essentially any RNA can be converted into a double stranded DNA suitable for restriction digestion, PCR expansion and sequencing using reverse transcriptase and a polymerase (“Reverse Transcription-PCR, or “RT-PCR”). See also, Ausubel, Sambrook and Berger, above. These methods can also be used to quantitatively amplify mRNA or corresponding cDNA, providing an indication of expression levels of mRNA that correspond to the genes or gene products of Tables 1-14 in an individual. Differences in expression levels for these genes between individuals, families, lines and/or populations can be used as markers for metabolic syndrome, insulin resistance, obesity, high blood pressure, dyslipidemia, diabetes and/or myocardial infarction.

Real Time Amplification/Detection Methods

In one aspect, real time PCR or LCR is performed on the amplification mixtures described herein, e.g., using molecular beacons or TaqMan™ probes. A molecular beacon (MB) is an oligonucleotide or PNA which, under appropriate hybridization conditions, self-hybridizes to form a stem and loop structure. The MB has a label and a quencher at the termini of the oligonucleotide or PNA; thus, under conditions that permit intra-molecular hybridization, the label is typically quenched (or at least altered in its fluorescence) by the quencher. Under conditions where the MB does not display intra-molecular hybridization (e.g., when bound to a target nucleic acid, e.g., to a region of an amplicon during amplification), the MB label is unquenched. Details regarding standard methods of making and using MBs are well established in the literature and MBs are available from a number of commercial reagent sources. See also, e.g., Leone et al. (1995) “Molecular beacon probes combined with amplification by NASBA enable homogenous real-time detection of RNA.” Nucleic Acids Res. 26:2150-2155; Tyagi and Kramer (1996) “Molecular beacons: probes that fluoresce upon hybridization” Nature Biotechnology 14:303-308; Blok and Kramer (1997) “Amplifiable hybridization probes containing a molecular switch” Mol Cell Probes 11:187-194; Hsuih et al. (1997) “Novel, ligation-dependent PCR assay for detection of hepatitis C in serum” J Clin Microbiol 34:501-507; Kostrikis et al. (1998) “Molecular beacons: spectral genotyping of human alleles” Science 279:1228-1229; Sokol et al. (1998) “Real time detection of DNA:RNA hybridization in living cells” Proc. Natl. Acad. Sci. U.S.A. 95:11538-11543; Tyagi et al. (1998) “Multicolor molecular beacons for allele discrimination” Nature Biotechnology 16:49-53; Bonnet et al. (1999) “Thermodynamic basis of the chemical specificity of structured DNA probes” Proc. Natl. Acad. Sci. U.S.A. 96:6171-6176; Fang et al. (1999) “Designing a novel molecular beacon for surface-immobilized DNA hybridization studies” J. Am. Chem. Soc. 121:2921-2922; Marras et al. (1999) “Multiplex detection of single-nucleotide variation using molecular beacons” Genet. Anal. Biomol. Eng. 14:151-156; and Vet et al. (1999) “Multiplex detection of four pathogenic retroviruses using molecular beacons” Proc. Natl. Acad. Sci. U.S.A. 96:6394-6399. Additional details regarding MB construction and use is found in the patent literature, e.g., U.S. Pat. No. 5,925,517 (Jul. 20, 1999) to Tyagi et al. entitled “Detectably labeled dual conformation oligonucleotide probes, assays and kits;” U.S. Pat. No. 6,150,097 to Tyagi et al (Nov. 21, 2000) entitled “Nucleic acid detection probes having non-FRET fluorescence quenching and kits and assays including such probes” and U.S. Pat. No. 6,037,130 to Tyagi et al (Mar. 14, 2000), entitled “Wavelength-shifting probes and primers and their use in assays and kits.”

PCR detection and quantification using dual-labeled fluorogenic oligonucleotide probes, commonly referred to as “TaqMan™” probes, can also be performed according to the present invention. These probes are composed of short (e.g., 20-25 base) oligodeoxynucleotides that are labeled with two different fluorescent dyes. On the 5′ terminus of each probe is a reporter dye, and on the 3′ terminus of each probe a quenching dye is found. The oligonucleotide probe sequence is complementary to an internal target sequence present in a PCR amplicon. When the probe is intact, energy transfer occurs between the two fluorophores and emission from the reporter is quenched by the quencher by FRET. During the extension phase of PCR, the probe is cleaved by 5′ nuclease activity of the polymerase used in the reaction, thereby releasing the reporter from the oligonucleotide-quencher and producing an increase in reporter emission intensity. Accordingly, TaqMan™ probes are oligonucleotides that have a label and a quencher, where the label is released during amplification by the exonuclease action of the polymerase used in amplification. This provides a real time measure of amplification during synthesis. A variety of TaqMan™ reagents are commercially available, e.g., from Applied Biosystems (Division Headquarters in Foster City, Calif.) as well as from a variety of specialty vendors such as Biosearch Technologies (e.g., black hole quencher probes). Further details regarding dual-label probe strategies can be found, e.g., in WO92/02638.

Other similar methods include e.g. fluorescence resonance energy transfer between two adjacently hybridized probes, e.g., using the “LightCycler®” format described in U.S. Pat. No. 6,174,670.

Array-Based Marker Detection

Array-based detection can be performed using commercially available arrays, e.g., from Affymetrix (Santa Clara, Calif.) or other manufacturers. Reviews regarding the operation of nucleic acid arrays include Sapolsky et al. (1999) “High-throughput polymorphism screening and genotyping with high-density oligonucleotide arrays.” Genetic Analysis: Biomolecular Engineering 14:187-192; Lockhart (1998) “Mutant yeast on drugs” Nature Medicine 4:1235-1236; Fodor (1997) “Genes, Chips and the Human Genome.” FASEB Journal 11:A879; Fodor (1997) “Massively Parallel Genomics.” Science 277: 393-395; and Chee et al. (1996) “Accessing Genetic Information with High-Density DNA Arrays.” Science 274:610-614. Array based detection is a preferred method for identification markers of the invention in samples, due to the inherently high-throughput nature of array based detection.

A variety of probe arrays have been described in the literature and can be used in the context of the present invention for detection of markers that can be correlated to the phenotypes noted herein (metabolic syndrome, insulin resistance, obesity, high blood pressure, dyslipidemia, diabetes and/or myocardial infarction, etc.). For example, DNA probe array chips or larger DNA probe array wafers (from which individual chips would otherwise be obtained by breaking up the wafer) are used in one embodiment of the invention. DNA probe array wafers generally comprise glass wafers on which high density arrays of DNA probes (short segments of DNA) have been placed. Each of these wafers can hold, for example, approximately 60 million DNA probes that are used to recognize longer sample DNA sequences (e.g., from individuals or populations, e.g., that comprise markers of interest). The recognition of sample DNA by the set of DNA probes on the glass wafer takes place through DNA hybridization. When a DNA sample hybridizes with an array of DNA probes, the sample binds to those probes that are complementary to the sample DNA sequence. By evaluating to which probes the sample DNA for an individual hybridizes more strongly, it is possible to determine whether a known sequence of nucleic acid is present or not in the sample, thereby determining whether a marker found in the nucleic acid is present. One can also use this approach to perform ASH, by controlling the hybridization conditions to permit single nucleotide discrimination, e.g., for SNP identification and for genotyping a sample for one or more SNPs.

The use of DNA probe arrays to obtain allele information typically involves the following general steps: design and manufacture of DNA probe arrays, preparation of the sample, hybridization of sample DNA to the array, detection of hybridization events and data analysis to determine sequence. Preferred wafers are manufactured using a process adapted from semiconductor manufacturing to achieve cost effectiveness and high quality, and are available, e.g., from Affymetrix, Inc. of Santa Clara, Calif.

For example, probe arrays can be manufactured by light-directed chemical synthesis processes, which combine solid-phase chemical synthesis with photolithographic fabrication techniques as employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays can be synthesized simultaneously on a large glass wafer. This parallel process enhances reproducibility and helps achieve economies of scale.

Once fabricated, DNA probe arrays can be used to obtain data regarding presence and/or expression levels for markers of interest. The DNA samples may be tagged with biotin and/or a fluorescent reporter group by standard biochemical methods. The labeled samples are incubated with an array, and segments of the samples bind, or hybridize, with complementary sequences on the array. The array can be washed and/or stained to produce a hybridization pattern. The array is then scanned and the patterns of hybridization are detected by emission of light from the fluorescent reporter groups. Additional details regarding these procedures are found in the examples below. Because the identity and position of each probe on the array is known, the nature of the DNA sequences in the sample applied to the array can be determined. When these arrays are used for genotyping experiments, they can be referred to as genotyping arrays.

The nucleic acid sample to be analyzed is isolated, amplified and, typically, labeled with biotin and/or a fluorescent reporter group. The labeled nucleic acid sample is then incubated with the array using a fluidics station and hybridization oven. The array can be washed and or stained or counter-stained, as appropriate to the detection method. After hybridization, washing and staining, the array is inserted into a scanner, where patterns of hybridization are detected. The hybridization data are collected as light emitted from the fluorescent reporter groups already incorporated into the labeled nucleic acid, which is now bound to the probe array. Probes that most clearly match the labeled nucleic acid produce stronger signals than those that have mismatches. Since the sequence and position of each probe on the array are known, by complementarity, the identity of the nucleic acid sample applied to the probe array can be identified.

In one embodiment, two DNA samples may be differentially labeled and hybridized with a single set of the designed genotyping arrays. In this way two sets of data can be obtained from the same physical arrays. Labels that can be used include, but are not limited to, cychrome, fluorescein, or biotin (later stained with phycoerythrin-streptavidin after hybridization). Two-color labeling is described in U.S. Pat. No. 6,342,355, incorporated herein by reference in its entirety. Each array may be scanned such that the signal from both labels is detected simultaneously, or may be scanned twice to detect each signal separately.

Intensity data is collected by the scanner for all the markers for each of the individuals that are tested for presence of the marker. The measured intensities are a measure indicative of the amount of a particular marker present in the sample for a given individual (expression level and/or number of copies of the allele present in an individual, depending on whether genomic or expressed nucleic acids are analyzed). This can be used to determine whether the individual is homozygous or heterozygous for the marker of interest. The intensity data is processed to provide corresponding marker information for the various intensities.

Additional Details Regarding Amplified Variable Sequences, SSR, AFLP ASH, SNPs and Isozyme Markers

Amplified variable sequences refer to amplified sequences of the genome which exhibit high nucleic acid residue variability between members of the same species. All organisms have variable genomic sequences and each organism (with the exception of a clone) has a different set of variable sequences. Once identified, the presence of specific variable sequence can be used to predict phenotypic traits. Preferably, DNA from the genome serves as a template for amplification with primers that flank a variable sequence of DNA. The variable sequence is amplified and then sequenced.

Alternatively, self-sustained sequence replication can be used to identify genetic markers. Self-sustained sequence replication refers to a method of nucleic acid amplification using target nucleic acid sequences which are replicated exponentially, in vitro, under substantially isothermal conditions by using three enzymatic activities involved in retroviral replication: (1) reverse transcriptase, (2) Rnase H, and (3) a DNA-dependent RNA polymerase (Guatelli et al. (1990) Proc Natl Acad Sci USA 87:1874). By mimicking the retroviral strategy of RNA replication by means of cDNA intermediates, this reaction accumulates cDNA and RNA copies of the original target.

Amplified fragment length polymorphisms (AFLP) can also be used as genetic markers (Vos et al. (1995) Nucl Acids Res 23:4407). The phrase “amplified fragment length polymorphism” refers to selected restriction fragments which are amplified before or after cleavage by a restriction endonuclease. The amplification step allows easier detection of specific restriction fragments. AFLP allows the detection large numbers of polymorphic markers and has been used for genetic mapping (Becker et al. (1995) Mol Gen Genet 249:65; and Meksem et al. (1995) Mol Gen Genet 249:74).

Allele-specific hybridization (ASH) can be used to identify the genetic markers of the invention. ASH technology is based on the stable annealing of a short, single-stranded, oligonucleotide probe to a completely complementary single-strand target nucleic acid. Detection may be accomplished via an isotopic or non-isotopic label attached to the probe.

For each polymorphism, two or more different ASH probes are designed to have identical DNA sequences except at the polymorphic nucleotides. Each probe will have exact homology with one allele sequence so that the range of probes can distinguish all the known alternative allele sequences. Each probe is hybridized to the target DNA. With appropriate probe design and hybridization conditions, a single-base mismatch between the probe and target DNA will prevent hybridization. In this manner, only one of the alternative probes will hybridize to a target sample that is homozygous or homogenous for an allele. Samples that are heterozygous or heterogeneous for two alleles will hybridize to both of two alternative probes.

ASH markers are used as dominant markers where the presence or absence of only one allele is determined from hybridization or lack of hybridization by only one probe. The alternative allele may be inferred from the lack of hybridization. ASH probe and target molecules are optionally RNA or DNA; the target molecules are any length of nucleotides beyond the sequence that is complementary to the probe; the probe is designed to hybridize with either strand of a DNA target; the probe ranges in size to conform to variously stringent hybridization conditions, etc.

PCR allows the target sequence for ASH to be amplified from low concentrations of nucleic acid in relatively small volumes. Otherwise, the target sequence from genomic DNA is digested with a restriction endonuclease and size separated by gel electrophoresis. Hybridizations typically occur with the target sequence bound to the surface of a membrane or, as described in U.S. Pat. No. 5,468,613, the ASH probe sequence may be bound to a membrane.

In one embodiment, ASH data are typically obtained by amplifying nucleic acid fragments (amplicons) from genomic DNA using PCR, transferring the amplicon target DNA to a membrane in a dot-blot format, hybridizing a labeled oligonucleotide probe to the amplicon target, and observing the hybridization dots by autoradiography.

Single nucleotide polymorphisms (SNP) are markers that consist of a shared sequence differentiated on the basis of a single nucleotide. Typically, this distinction is detected by differential migration patterns of an amplicon comprising the SNP on e.g., an acrylamide gel. However, alternative modes of detection, such as hybridization, e.g., ASH, or RFLP analysis are also appropriate.

Isozyme markers can be employed as genetic markers, e.g., to track isozyme markers linked to the markers herein. Isozymes are multiple forms of enzymes that differ from one another in their amino acid, and therefore their nucleic acid sequences. Some isozymes are multimeric enzymes contain slightly different subunits. Other isozymes are either multimeric or monomeric but have been cleaved from the proenzyme at different sites in the amino acid sequence. Isozymes can be characterized and analyzed at the protein level, or alternatively, isozymes which differ at the nucleic acid level can be determined. In such cases any of the nucleic acid based methods described herein can be used to analyze isozyme markers.

Additional Details Regarding Nucleic Acid Amplification

As noted, nucleic acid amplification techniques such as PCR and LCR are well known in the art and can be applied to the present invention to amplify and/or detect nucleic acids of interest, such as nucleic acids comprising marker loci. Examples of techniques sufficient to direct persons of skill through such in vitro methods, including the polymerase chain reaction (PCR), the ligase chain reaction (LCR), Qβ-replicase amplification and other RNA polymerase mediated techniques (e.g., NASBA), are found in the references noted above, e.g., Innis, Sambrook, Ausubel, and Berger. Additional details are found in Mullis et al. (1987) U.S. Pat. No. 4,683,202; Arnheim & Levinson (Oct. 1, 1990) C&EN 36-47; The Journal Of NIH Research (1991) 3, 81-94; (Kwoh et al. (1989) Proc. Natl. Acad. Sci. USA 86, 1173; Guatelli et al. (1990) Proc. Natl. Acad. Sci. USA 87, 1874; Lomell et al. (1989) J. Clin. Chem 35, 1826; Landegren et al., (1988) Science 241, 1077-1080; Van Brunt (1990) Biotechnology 8, 291-294; Wu and Wallace, (1989) Gene 4, 560; Barringer et al. (1990) Gene 89, 117, and Sooknanan and Malek (1995) Biotechnology 13: 563-564. Improved methods of amplifying large nucleic acids by PCR, which is useful in the context of positional cloning, are further summarized in Cheng et al. (1994) Nature 369: 684, and the references therein, in which PCR amplicons of up to 40 kb are generated. Methods for long-range PCR are disclosed, for example, in U.S. patent application Ser. No. 10/042,406, filed Jan. 9, 2002, entitled “Algorithms for Selection of Primer Pairs”; U.S. patent application Ser. No. 10/236,480, filed Sep. 9, 2002, entitled “Methods for Amplification of Nucleic Acids”; and U.S. Pat. No. 6,740,510, issued May 25, 2004, entitled “Methods for Amplification of Nucleic Acids”. U.S. Ser. No. 10/341,832 (filed Jan. 14, 2003) also provides details regarding primer picking methods for performing short range PCR.

Detection of Protein Expression Products

Proteins such as those encoded by the genes noted in Tables 1-14, 17 and 18 are encoded by nucleic acids, including those comprising markers that are correlated to the phenotypes of interest herein. For a description of the basic paradigm of molecular biology, including the expression (transcription and/or translation) of DNA into RNA into protein, see, Alberts et al. (2002) Molecular Biology of the Cell, 4^{th} Edition Taylor and Francis, Inc., ISBN: 0815332181 (“Alberts”), and Lodish et al. (1999) Molecular Cell Biology, 4^{th} Edition W H Freeman & Co, ISBN: 071673706X (“Lodish”). Accordingly, proteins corresponding to the genes in Tables 1-14, 17 and 18 can be detected as markers, e.g., by detecting different protein isotypes between individuals or populations, or by detecting a differential presence, absence or expression level of such a protein of interest (e.g., expression level of a gene product of Tables 1-14, 17 and 18).

A variety of protein detection methods are known and can be used to distinguish markers. In addition to the various references noted supra, a variety of protein manipulation and detection methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2^{nd} Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, NY; Janson and Ryden (1998) Protein Purification: Principles High Resolution Methods and Applications, Second Edition Wiley-VCH, NY; and Walker (1998) Protein Protocols on CD-ROM Humana Press, NJ; and the references cited therein. Additional details regarding protein purification and detection methods can be found in Satinder Ahuja ed., Handbook of Bioseparations, Academic Press (2000).

“Proteomic” detection methods, which detect many proteins simultaneously have been described. These can include various multidimensional electrophoresis methods (e.g., 2-d gel electrophoresis), mass spectrometry based methods (e.g., SELDI, MALDI, electrospray, etc.), or surface plasmon reasonance methods. For example, in MALDI, a sample is usually mixed with an appropriate matrix, placed on the surface of a probe and examined by laser desorption/ionization. The technique of MALDI is well known in the art. See, e.g., U.S. Pat. No. 5,045,694 (Beavis et al.), U.S. Pat. No. 5,202,561 (Gleissmann et al.), and U.S. Pat. No. 6,111,251 (Hillenkamp). Similarly, for SELDI, a first aliquot is contacted with a solid support-bound (e.g., substrate-bound) adsorbent. A substrate is typically a probe (e.g., a biochip) that can be positioned in an interrogatable relationship with a gas phase ion spectrometer. SELDI is also a well known technique, and has been applied to diagnostic proteomics. See, e.g. Issaq et al. (2003) “SELDI-TOF MS for Diagnostic Proteomics” Analytical Chemistry 75:149A-155A.

In general, the above methods can be used to detect different forms (alleles) of proteins and/or can be used to detect different expression levels of the proteins (which can be due to allelic differences) between individuals, families, lines, populations, etc. Differences in expression levels, when controlled for environmental factors, can be indicative of different alleles at a QTL for the gene of interest, even if the encoded differentially expressed proteins are themselves identical. This occurs, for example, where there are multiple allelic forms of a gene in non-coding regions, e.g., regions such as promoters or enhancers that control gene expression. Thus, detection of differential expression levels can be used as a method of detecting allelic differences.

In other aspects of the present invention, a gene comprising, in linkage disequilibrium with, or under the control of a nucleic acid associated with metabolic syndrome, insulin resistance, obesity, high blood pressure, dyslipidemia, diabetes and/or myocardial infarction may exhibit differential allelic expression. “Differential allelic expression” as used herein refers to both qualitative and quantitative differences in the allelic expression of multiple alleles of a single gene present in a cell. As such, a gene displaying differential allelic expression may have one allele expressed at a different time or level as compared to a second allele in the same cell/tissue. For example, an allele associated with metabolic syndrome may be expressed at a higher or lower level than an allele that is not associated with metabolic syndrome, even though both are alleles of the same gene and are present in the same cell/tissue. Differential allelic expression and analysis methods are disclosed in detail in U.S. patent application Ser. No. 10/438,184, filed May 13, 2003 and U.S. patent application Ser. No. 10/845,316, filed May 12, 2004, both of which are entitled “Allele-specific expression patterns.” Detection of a differential allelic expression pattern of one or more nucleic acids, or fragments, derivatives, polymorphisms, variants or complements thereof, associated with susceptibility to metabolic syndrome, insulin resistance, obesity, high blood pressure, dyslipidemia, diabetes and/or myocardial infarction is a prognostic and diagnostic for susceptibility to metabolic syndrome, insulin resistance, obesity, high blood pressure, dyslipidemia, diabetes and/or myocardial infarction, respectively; likewise, detection of a differential allelic expression pattern of one or more nucleic acids, or fragments, derivatives, polymorphisms, variants or complements thereof, associated with resistance to metabolic syndrome, insulin resistance, obesity, high blood pressure, dyslipidemia, diabetes and/or myocardial infarction is a prognostic and diagnostic for resistance to metabolic syndrome, insulin resistance, obesity, high blood pressure, dyslipidemia, diabetes and/or myocardial infarction, respectively.

Additional Details Regarding Types of Markers Appropriate for Screening

The biological markers that are screened for correlation to the phenotypes herein can be any of those types of markers that can be detected by screening, e.g., genetic markers such as allelic variants of a genetic locus (e.g., as in SNPs), expression markers (e.g., presence or quantity of mRNAs and/or proteins), and/or the like.

The nucleic acid of interest to be amplified, transcribed, translated and/or detected in the methods of the invention can be essentially any nucleic acid, though nucleic acids derived from human sources are especially relevant to the detection of markers associated with disease diagnosis and clinical applications. The sequences for many nucleic acids and amino acids (from which nucleic acid sequences can be derived via reverse translation) are available, including for the genes or gene products of Tables 1-14, 17 and 18. Common sequence repositories for known nucleic acids include GenBank® EMBL, DDBJ and the NCBI. Other repositories can easily be identified by searching the internet. The nucleic acid to be amplified, transcribed, translated and/or detected can be an RNA (e.g., where amplification includes RT-PCR or LCR, the Van-Gelder Eberwine reaction or Ribo-SPIA) or DNA (e.g., amplified DNA, cDNA or genomic DNA), or even any analogue thereof (e.g., for detection of synthetic nucleic acids or analogues thereof, e.g., where the sample of interest includes or is used to derive or synthesize artificial nucleic acids). Any variation in a nucleic acid sequence or expression level between individuals or populations can be detected as a marker, e.g., a mutation, a polymorphism, a single nucleotide polymorphism (SNP), an allele, an isotype, expression of an RNA or protein, etc. One can detect variation in sequence, expression levels or gene copy numbers as markers that can be correlated to metabolic syndrome, insulin resistance, obesity, high blood pressure, dyslipidemia, diabetes and/or myocardial infarction.

For example, the methods of the invention are useful in screening samples derived from patients for a marker nucleic acid of interest, e.g., from bodily fluids (blood, saliva, urine etc.), tissue, and/or waste from the patient. Thus, stool, sputum, saliva, blood, lymph, tears, sweat, urine, vaginal secretions, ejaculatory fluid or the like can easily be screened for nucleic acids by the methods of the invention, as can essentially any tissue of interest that contains the appropriate nucleic acids. These samples are typically taken, following informed consent, from a patient by standard medical laboratory methods.

Prior to amplification and/or detection of a nucleic acid comprising a marker, the nucleic acid is optionally purified from the samples by any available method, e.g., those taught in Berger and Kimmel, Guide to Molecular Cloning Techniques Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2001 (“Sambrook”); and/or Current Protocols in Molecular Biology, F. M. Ausubel et al., eds., Current Protocols, a joint venture between Greene Publishing Associates, Inc. and John Wiley & Sons, Inc., (supplemented through 2002) (“Ausubel”)). A plethora of kits are also commercially available for the purification of nucleic acids from cells or other samples (see, e.g., EasyPrep™, FlexiPrep™, both from Pharmacia Biotech; StrataClean™, from Stratagene; and, QIAprep™ from Qiagen). Alternately, samples can simply be directly subjected to amplification or detection, e.g., following aliquotting and/or dilution.

Examples of markers can include polymorphisms, single nucleotide polymorphisms, presence of one or more nucleic acids in a sample, absence of one or more nucleic acids in a sample, presence of one or more genomic DNA sequences, absence or one or more genomic DNA sequences, presence of one or more mRNAs, absence of one or more mRNAs, expression levels of one or more mRNAs, presence of one or more proteins, expression levels of one or more proteins, and/or data derived from any of the preceding or combinations thereof. Essentially any number of markers can be detected, using available methods, e.g., using array technologies that provide high density, high throughput marker mapping. Thus, at least about 10, 20, 50, 100, 1,000, 10,000, or even 100,000 or more genetic markers can be tested, simultaneously or in a serial fashion (or combination thereof), for correlation to a relevant phenotype, in the first and/or second population. Combinations of markers can also be desirably tested, e.g., to identify genetic combinations or combinations of expression patterns in populations that are correlated to the phenotype.

As noted, the biological marker to be detected can be any detectable biological component. Commonly detected markers include genetic markers (e.g., DNA sequence markers present in genomic DNA or expression products thereof) and expression markers (which can reflect genetically coded factors, environmental factors, or both). Where the markers are expression markers, the methods can include determining a first expression profile for a first individual or population (e.g., of one or more expressed markers, e.g., a set of expressed markers) and comparing the first expression profile to a second expression profile for the second individual or population. In this example, correlating expression marker(s) to a particular phenotype can include correlating the first or second expression profile to the phenotype of interest.

Probe/Primer Synthesis Methods

In general, synthetic methods for making oligonucleotides, including probes, primers, molecular beacons, PNAs, LNAs (locked nucleic acids), etc., are well known. For example, oligonucleotides can be synthesized chemically according to the solid phase phosphoramidite triester method described by Beaucage and Caruthers (1981), Tetrahedron Letts., 22(20):1859-1862, e.g., using a commercially available automated synthesizer, e.g., as described in Needham-VanDevanter et al. (1984) Nucleic Acids Res., 12:6159-6168. Oligonucleotides, including modified oligonucleotides can also be ordered from a variety of commercial sources known to persons of skill. There are many commercial providers of oligo synthesis services, and thus this is a broadly accessible technology. Any nucleic acid can be custom ordered from any of a variety of commercial sources, such as The Midland Certified Reagent Company (mcrc@oligos.com), The Great American Gene Company (www.genco.com), ExpressGen Inc. (www.expressgen.com), Operon Technologies Inc. (Alameda, Calif.) and many others. Similarly, PNAs can be custom ordered from any of a variety of sources, such as PeptidoGenic (pkim@ccnet.com), HTI Bio-products, inc. (htibio.com), BMA Biomedicals Ltd (U.K.), Bio-Synthesis, Inc., and many others.

In Silico Marker Detection

In some embodiments, in silico methods can be used to detect the marker loci of interest. For example, the sequence of a nucleic acid comprising the marker locus of interest can be stored in a computer. The desired marker locus sequence or its homolog can be identified using an appropriate nucleic acid search algorithm as provided by, for example, in such readily available programs as BLAST, or even simple word processors. The entire human genome has been sequenced and, thus, sequence information can be used to identify marker regions, flanking nucleic acids, etc.

Amplification Primers for Marker Detection

In some preferred embodiments, the molecular markers of the invention are detected using a suitable PCR-based detection method, where the size or sequence of the PCR amplicon is indicative of the absence or presence of the marker (e.g., a particular marker allele). In these types of methods, PCR primers are hybridized to the conserved regions flanking the polymorphic marker region.

It will be appreciated that suitable primers to be used with the invention can be designed using any suitable method. It is not intended that the invention be limited to any particular primer or primer pair. For example, primers can be designed using any suitable software program, such as LASERGENE®, e.g., taking account of publicly available sequence information.

In some embodiments, the primers of the invention are radiolabelled, or labeled by any suitable means (e.g., using a non-radioactive fluorescent tag), to allow for rapid visualization of the different size amplicons following an amplification reaction without any additional labeling step or visualization step. In some embodiments, the primers are not labeled, and the amplicons are visualized following their size resolution, e.g., following agarose or acrylamide gel electrophoresis. In some embodiments, ethidium bromide staining of the PCR amplicons following size resolution allows visualization of the different size amplicons.

It is not intended that the primers of the invention be limited to generating an amplicon of any particular size. For example, the primers used to amplify the marker loci and alleles herein are not limited to amplifying the entire region of the relevant locus. The primers can generate an amplicon of any suitable length that is longer or shorter than those given as example amplicons in Tables 1-14, 17 and 18. In some embodiments, marker amplification produces an amplicon at least 20 nucleotides in length, or alternatively, at least 50 nucleotides in length, or alternatively, at least 100 nucleotides in length, or alternatively, at least 200 nucleotides in length.

Detection of Markers for Positional Cloning

In some embodiments, a nucleic acid probe is used to detect a nucleic acid that comprises a marker sequence. Such probes can be used, for example, in positional cloning to isolate nucleotide sequences linked to the marker nucleotide sequence. It is not intended that the nucleic acid probes of the invention be limited to any particular size. In some embodiments, nucleic acid probe is at least 20 nucleotides in length, or alternatively, at least 50 nucleotides in length, or alternatively, at least 100 nucleotides in length, or alternatively, at least 200 nucleotides in length.

A hybridized probe is detected using, autoradiography, fluorography or other similar detection techniques depending on the label to be detected. Examples of specific hybridization protocols are widely available in the art, see, e.g., Berger, Sambrook, and Ausubel, all herein.

Generation of Transgenic Cells and Organisms

The present invention also provides cells and organisms which are transformed with nucleic acids corresponding to QTL identified according to the invention. For example, such nucleic acids include chromosome intervals (e.g., genomic fragments), ORFs and/or cDNAs that encode genes that correspond or are linked to QTL for metabolic syndrome, insulin resistance, obesity, high blood pressure, dyslipidemia, diabetes and/or myocardial infarction. Additionally, the invention provides for the production of polypeptides that influence metabolic syndrome, insulin resistance, obesity, high blood pressure, dyslipidemia, diabetes and/or myocardial infarction. This is useful, e.g., to influence metabolic syndrome, insulin resistance, obesity, high blood pressure, dyslipidemia, diabetes and/or myocardial infarction in livestock populations. The generation of transgenic cells also provides commercially useful cells having defined genes that influence phenotype, thereby providing a platform for screening potential modulators of phenotype, as well as basic research into the mechanism of action for each of the genes of interest. In addition, gene therapy can be used to introduce desirable genes into individuals or populations thereof. Such gene therapies may be used to provide a treatment for a disorder exhibited by an individual, or may be used as a preventative measure to prevent the development of such a disorder in an individual at risk. Knock-out animals, such as knock-out mice, can be produced for any of the genes noted herein, to further identify phenotypic effects of the genes. Similarly, recombinant mice or other animals can be used as models for human disease, e.g., by knocking out any natural gene herein and introduction (e.g., via homologous recombination) of the human (or other species) gene into the animal. The effects of modulators on the heterologous human genes and gene products can then be monitored in the resulting in vivo model animal system.

General texts which describe molecular biological techniques for the cloning and manipulation of nucleic acids and production of encoded polypeptides include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2001 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 2004 or later) (“Ausubel”)). These texts describe mutagenesis, the use of vectors, promoters and many other relevant topics related to, e.g., the generation of clones that comprise nucleic acids of interest, e.g., genes, marker loci, marker probes, QTL that segregate with marker loci, etc.

Host cells are genetically engineered (e.g., transduced, transfected, transformed, etc.) with the vectors of this invention (e.g., vectors, such as expression vectors which comprise an ORF derived from or related to a QTL) which can be, for example, a cloning vector, a shuttle vector or an expression vector. Such vectors are, for example, in the form of a plasmid, a phagemid, an agrobacterium, a virus, a naked polynucleotide (linear or circular), or a conjugated polynucleotide. Vectors can be introduced into bacteria, especially for the purpose of propagation and expansion. Additional details regarding nucleic acid introduction methods are found in Sambrook, Berger and Ausubel, infra. The method of introducing a nucleic acid of the present invention into a host cell is not critical to the instant invention, and it is not intended that the invention be limited to any particular method for introducing exogenous genetic material into a host cell. Thus, any suitable method, e.g., including but not limited to the methods provided herein, which provides for effective introduction of a nucleic acid into a cell or protoplast can be employed and finds use with the invention.

The engineered host cells can be cultured in conventional nutrient media modified as appropriate for such activities as, for example, activating promoters or selecting transformants. In addition to Sambrook, Berger and Ausubel, all infra, Atlas and Parks (eds) The Handbook of Microbiological Media (1993) CRC Press, Boca Raton, Fla. and available commercial literature such as the Life Science Research Cell Culture Catalogue (2004) from Sigma-Aldrich, Inc (St Louis, Mo.) (“Sigma-LSRCCC”) provide additional details.

Making Knock-Out Animals and Transgenics

Transgenic animals are a useful tool for studying gene function and testing putative gene or gene product modulators. Human (or other selected species) genes herein can be introduced in place of endogenous genes of a laboratory animal, making it possible to study function of the human (or other, e.g., livestock) gene or gene product in the easily manipulated and studied laboratory animal.

It will be appreciated that there is not always a precise correspondence for responses to modulators between homologous gene in different animals, making the ability to study the human or other species of interest (e.g., a livestock species) in a laboratory animal particularly useful. Although similar genetic manipulations can be performed in tissue culture, the interaction of genes and gene products in the context of an intact organism provides a more complete and physiologically relevant picture of such genes and gene products than can be achieved in simple cell-based screening assays. Accordingly, one feature of the invention is the creation of transgenic animals comprising heterologous genes of interest, e.g., genes listed in Tables 1-14, 17 and 18.

In general, such a transgenic animal is simply an animal that has had appropriate genes (or partial genes, e.g., comprising coding sequences coupled to a promoter) introduced into one or more of its cells artificially. This is most commonly done in one of two ways. First, a DNA can be integrated randomly by injecting it into the pronucleus of a fertilized ovum. In this case, the DNA can integrate anywhere in the genome. In this approach, there is no need for homology between the injected DNA and the host genome. Second, targeted insertion can be accomplished by introducing the (heterologous) DNA into embryonic stem (ES) cells and selecting for cells in which the heterologous DNA has undergone homologous recombination with homologous sequences of the cellular genome. Typically, there are several kilobases of homology between the heterologous and genomic DNA, and positive selectable markers (e.g., antibiotic resistance genes) are included in the heterologous DNA to provide for selection of transformants. In addition, negative selectable markers (e.g., “toxic” genes such as barnase) can be used to select against cells that have incorporated DNA by non-homologous recombination (random insertion).

One common use of targeted insertion of DNA is to make knock-out mice. Typically, homologous recombination is used to insert a selectable gene driven by a constitutive promoter into an essential exon of the gene that one wishes to disrupt (e.g., the first coding exon). To accomplish this, the selectable marker is flanked by large stretches of DNA that match the genomic sequences surrounding the desired insertion point. Once this construct is electroporated into ES cells, the cells' own machinery performs the homologous recombination. To make it possible to select against ES cells that incorporate DNA by non-homologous recombination, it is common for targeting constructs to include a negatively selectable gene outside the region intended to undergo recombination (typically the gene is cloned adjacent to the shorter of the two regions of genomic homology). Because DNA lying outside the regions of genomic homology is lost during homologous recombination, cells undergoing homologous recombination cannot be selected against, whereas cells undergoing random integration of DNA often can. A commonly used gene for negative selection is the herpes virus thymidine kinase gene, which confers sensitivity to the drug gancyclovir.

Following positive selection and negative selection if desired, ES cell clones are screened for incorporation of the construct into the correct genomic locus. Typically, one designs a targeting construct so that a band normally seen on a Southern blot or following PCR amplification becomes replaced by a band of a predicted size when homologous recombination occurs. Since ES cells are diploid, only one allele is usually altered by the recombination event so, when appropriate targeting has occurred, one usually sees bands representing both wild type and targeted alleles.

The embryonic stem (ES) cells that are used for targeted insertion are derived from the inner cell masses of blastocysts (early mouse embryos). These cells are pluripotent, meaning they can develop into any type of tissue.

Once positive ES clones have been grown up and frozen, the production of transgenic animals can begin. Donor females are mated, blastocysts are harvested, and several ES cells are injected into each blastocyst. Blastocysts are then implanted into a uterine horn of each recipient. By choosing an appropriate donor strain, the detection of chimeric offspring (i.e., those in which some fraction of tissue is derived from the transgenic ES cells) can be as simple as observing hair and/or eye color. If the transgenic ES cells do not contribute to the germline (sperm or eggs), the transgene cannot be passed on to offspring.

Correlating Markers to Phenotypes

One aspect of the invention is a description of correlations between polymorphisms within or linked to the genes and polymorphisms noted in Tables 1-14 and metabolic syndrome phenotypes. Specifically, Table 1 lists genes and polymorphisms that are associated with metabolic syndrome; Table 2 lists genes and polymorphisms that are associated with diabetes; Table 3 lists genes and polymorphisms that are associated with myocardial infarction; Table 4 lists genes and polymorphisms that are associated with hypertension; Table 5 lists genes and polymorphisms that are associated with body mass index; Table 6 lists genes and polymorphisms that are associated with waist circumference; Table 7 lists genes and polymorphisms that are associated with diastolic blood pressure; Table 8 lists genes and polymorphisms that are associated with systolic blood pressure; Table 9 lists genes and polymorphisms that are associated with HDL cholesterol levels; Table 10 lists genes and polymorphisms that are associated with triglyceride levels; Table 11 lists genes and polymorphisms that are associated with insulin levels in nondiabetics; Table 12 lists genes and polymorphisms that are associated with insulin levels in non-Mexicans; Table 13 lists genes and polymorphisms that are associated with insulin resistance in nondiabetics; and Table 14 lists genes and polymorphisms that are associated with insulin resistance in non-Mexicans. Further details about these metabolic syndrome phenotypes is provided in Example 10, below. An understanding of these correlations can be used in the present invention to correlate information regarding a set of polymorphisms that an individual or sample is determined to possess and a phenotype that they are likely to display. Further, higher order correlations that account for combinations of alleles in one or more different genes can also be assessed for correlations to phenotype.

These correlations can be performed by any method that can identify a relationship between an allele and a phenotype, or a combination of alleles and a combination of phenotypes. For example, alleles in one or more of the genes or loci in Tables 1-14 can be correlated with one or more metabolic syndrome, insulin resistance, obesity, high blood pressure, dyslipidemia, diabetes and/or myocardial infarction phenotypes. Most typically, these methods involve referencing a look up table that comprises correlations between alleles of the polymorphism and the phenotype. The table can include data for multiple allele-phenotype relationships and can take account of additive or other higher order effects of multiple allele-phenotype relationships, e.g., through the use of statistical tools such as principle component analysis, heuristic algorithms, etc.

Correlation of a marker to a phenotype optionally includes performing one or more statistical tests for correlation. Many statistical tests are known, and most are computer-implemented for ease of analysis. A variety of statistical methods of determining associations/correlations between phenotypic traits and biological markers are known and can be applied to the present invention. For an introduction to the topic, see, Hartl (1981) A Primer of Population Genetics Washington University, Saint Louis Sinauer Associates, Inc. Sunderland, Mass. ISBN: 0-087893-271-2. A variety of appropriate statistical models are described in Lynch and Walsh (1998) Genetics and Analysis of Quantitative Traits, Sinauer Associates, Inc. Sunderland Mass. ISBN 0-87893-481-2. These models can, for example, provide for correlations between genotypic and phenotypic values, characterize the influence of a locus on a phenotype, sort out the relationship between environment and genotype, determine dominance or penetrance of genes, determine maternal and other epigenetic effects, determine principle components in an analysis (via principle component analysis, or “PCA”), and the like. The references cited in these texts provides considerable further detail on statistical models for correlating markers and phenotype.

In addition to standard statistical methods for determining correlation, other methods that determine correlations by pattern recognition and training, such as the use of genetic algorithms, can be used to determine correlations between markers and phenotypes. This is particularly useful when identifying higher order correlations between multiple alleles and multiple phenotypes. To illustrate, neural network approaches can be coupled to genetic algorithm-type programming for heuristic development of a structure-function data space model that determines correlations between genetic information and phenotypic outcomes. For example, NNUGA (Neural Network Using Genetic Algorithms) is an available program (e.g., on the world wide web at cs.bgu.ac.il/˜omri/NNUGA which couples neural networks and genetic algorithms. An introduction to neural networks can be found, e.g., in Kevin Gurney, An Introduction to Neural Networks, UCL Press (1999) and on the world wide web at shef.ac.uk/psychology/gurney/notes/index.html. Additional useful neural network references include those noted above in regard to genetic algorithms and, e.g., Bishop, Neural Networks for Pattern Recognition, Oxford University Press (1995), and Ripley et al., Pattern Recognition and Neural Networks, Cambridge University Press (1995).

Additional references that are useful in understanding data analysis applications for using and establishing correlations, principle components of an analysis, neural network modeling and the like, include, e.g., Hinchliffe, Modeling Molecular Structures, John Wiley and Sons (1996), Gibas and Jambeck, Bioinformatics Computer Skills, O'Reilly (2001), Pevzner, Computational Molecular Biology and Algorithmic Approach, The MIT Press (2000), Durbin et al., Biological Sequence Analysis: Probabilistic Models of Proteins and Nucleic Acids, Cambridge University Press (1998), and Rashidi and Buehler, Bioinformatic Basics: Applications in Biological Science and Medicine, CRC Press LLC (2000).

In any case, essentially any statistical test can be applied in a computer implemented model, by standard programming methods, or using any of a variety of “off the shelf” software packages that perform such statistical analyses, including, for example, those noted above and those that are commercially available, e.g., from Partek Incorporated (St. Peters, Mo.; www(dot)partek(dot)com), e.g., that provide software for pattern recognition (e.g., which provide Partek Pro 2000 Pattern Recognition Software) which can be applied to genetic algorithms for multivariate data analysis, interactive visualization, variable selection, neural network & statistical modeling, etc. Relationships can be analyzed, e.g., by Principal Components Analysis (PCA) mapped mapped scatterplots and biplots, Multi-Dimensional Scaling (MDS) Multi-Dimensional Scaling (MDS) mapped scatterplots, star plots, etc. Available software for performing correlation analysis includes SAS, R and MathLab.

In any case, the marker(s), whether polymorphisms or expression patterns, can be used for any of a variety of genetic analyses. For example, once markers have been identified, as in the present case, they can be used in a number of different assays for association studies. For example, probes can be designed for microarrays that interrogate these markers. Other exemplary assays include, e.g., the Taqman assays and molecular beacon assays described supra, as well as conventional PCR and/or sequencing techniques.

Additional details regarding association studies can be found in U.S. Pat. No. 6,969,589; U.S. Pat. No. 6,897,025; U.S. Ser. No. 10/286,417, filed Oct. 31, 2002, entitled “Methods for Genomic Analysis;” U.S. Ser. No. 10/768,788, filed Jan. 30, 2004, entitled “Apparatus and Methods for Analyzing and Characterizing Nucleic Acid Sequences;” U.S. Ser. No. 10/447,685, filed May 28, 2003, entitled “Liver Related Disease Compositions and Methods;” U.S. Ser. No. 10/970,761, filed Oct. 20, 2004, entitled “Improved Analysis Methods and Apparatus for Individual Genotyping” (methods for individual genotyping); U.S. Ser. No. 10/956,224, filed Sep. 30, 2004, entitled “Methods for Genetic Analysis;” U.S. Ser. No. 11/172,341, filed Jun. 29, 2005, entitled “Methods for Genomic Analysis;” U.S. Ser. No. 11/344,975, filed Jan. 31, 2006, entitled “Genetic Basis of Alzheimer's Disease and Diagnosis and Treatment Thereof;” and U.S. Ser. No. 11/043,689, filed Jan. 24, 2005, entitled “Associations Using Genotypes and Phenotypes,” and Aguilar-Salinas, et al. (2006) “Design and Validation of a Population-Based Definition of the Metabolic Syndrome” Diabetes Care 29(11):2420-2426.

In some embodiments, the marker data is used to perform association studies to show correlations between markers and phenotypes. This can be accomplished by determining marker characteristics in individuals with the phenotype of interest (i.e., individuals or populations displaying the phenotype of interest) and comparing the allele frequency or other characteristics (expression levels, etc.) of the markers in these individuals to the allele frequency or other characteristics in a control group of individuals. Such marker determinations can be conducted on a genome-wide basis, or can be focused on specific regions of the genome (e.g., haplotype blocks of interest). In one embodiment, markers that are linked to the genes or loci in Tables 1-14 are assessed for correlation to one or more specific phenotypes.

In addition to the other embodiments of the methods of the present invention disclosed herein, the methods additionally allow for the “dissection” of a phenotype. That is, a particular phenotype can result from two or more different genetic bases. For example, a metabolic syndrome phenotype in one individual may be the result of “defects” (or simply particular alleles—“defect” with respect to a susceptibility phenotype is context dependent, e.g., whether the phenotype is desirable or undesirable in the individual in a given environment) in a subset of genes in Tables 1-14, while the same basic phenotype in a different individual may be the result of multiple “defects” in a different subset of genes listed in Tables 1-14. Thus, scanning a plurality of markers (e.g., as in genome or haplotype block scanning) allows for the dissection of varying genetic bases for similar (or graduated) phenotypes.

As described in the previous paragraph, one method of conducting association studies is to compare the allele frequency (or expression level) of markers in individuals with a phenotype of interest (“case group”) to the allele frequency in a control group of individuals. In one method, informative SNPs are used to make the SNP haplotype pattern comparison (an “informative SNP” is genetic SNP marker such as a SNP or subset (more than one) of SNPs in a genome or haplotype block that tends to distinguish one SNP or genome or haplotype pattern from other SNPs, genomes or haplotype patterns; informative SNPs may also be referred to as “tag SNPs”). The approach of using informative SNPs has an advantage over other whole genome scanning or genotyping methods known in the art, for instead of reading all 3 billion bases of each individual's genome—or even reading the 3-4 million common SNPs that may be found-only informative SNPs from a sample population need to be detected. Reading these particular, informative SNPs provides sufficient information to allow statistically accurate association data to be extracted from specific experimental populations, as described above.

Thus, in an embodiment of one method of determining genetic associations, the allele frequency of informative SNPs is determined for genomes of a control population that do not display the phenotype. The allele frequency of informative SNPs is also determined for genomes of a population that do display the phenotype. The informative SNP allele frequencies are compared. Allele frequency comparisons can be made, for example, by determining the allele frequency (number of instances of a particular allele in a population divided by the total number of alleles) at each informative SNP location in each population and comparing these allele frequencies. The informative SNPs displaying a difference between the allele frequency of occurrence in the control versus case populations/groups are selected for analysis. Once informative SNPs are selected, the SNP haplotype block(s) that contain the informative SNPs are identified, which in turn identifies a genomic region of interest that is correlated with the phenotype. The genomic regions can be analyzed by genetic or any biological methods known in the art e.g., for use as drug discovery targets or as diagnostic markers.

### Systems for Identifying a Metabolic Syndrome Phenotype

Systems for performing the above correlations are also a feature of the invention. Typically, the system will include system instructions that correlate the presence or absence of an allele (whether detected directly or, e.g., through expression analysis) with a predicted metabolic syndrome, insulin resistance, obesity, high blood pressure, dyslipidemia, diabetes and/or myocardial infarction phenotype. The system instructions can compare detected information as to allele sequence or expression level with a database that includes correlations between the alleles and the relevant phenotypes. As noted above, this database can be multidimensional, thereby including higher-order relationships between combinations of alleles and the relevant phenotypes. These relationships can be stored in any number of look-up tables, e.g., taking the form of spreadsheets (e.g., Excel™ spreadsheets) or databases such as an Access™, SQL™, Oracle™, Paradox™, or similar database. The system includes provisions for inputting sample-specific information regarding allele detection information, e.g., through an automated or user interface and for comparing that information to the look up tables.

Optionally, the system instructions can also include software that accepts diagnostic information associated with any detected allele information, e.g., a diagnosis that a subject with the relevant allele has a particular phenotype (e.g., a metabolic syndrome phenotype, such as metabolic syndrome, insulin resistance, obesity, high blood pressure, dyslipidemia, diabetes and/or myocardial infarction). This software can be heuristic in nature, using such inputted associations to improve the accuracy of the look up tables and/or interpretation of the look up tables by the system. A variety of such approaches, including neural networks, Markov modeling, and other statistical analysis are described above.

The invention provides data acquisition modules for detecting one or more detectable genetic marker(s) (e.g., one or more array comprising one or more biomolecular probes, detectors, fluid handlers, or the like). The biomolecular probes of such a data acquisition module can include any that are appropriate for detecting the biological marker, e.g., oligonucleotide probes, proteins, aptamers, antibodies, etc. These can include sample handlers (e.g., fluid handlers), robotics, microfluidic systems, nucleic acid or protein purification modules, arrays (e.g., nucleic acid arrays), detectors, thermocyclers or combinations thereof, e.g., for acquiring samples, diluting or aliquotting samples, purifying marker materials (e.g., nucleic acids or proteins), amplifying marker nucleic acids, detecting amplified marker nucleic acids, and the like.

For example, automated devices that can be incorporated into the systems herein have been used to assess a variety of biological phenomena, including, e.g., expression levels of genes in response to selected stimuli (Service (1998) “Microchips Arrays Put DNA on the Spot” Science 282:396-399), high throughput DNA genotyping (Zhang et al. (1999) “Automated and Integrated System for High-Throughput DNA Genotyping Directly from Blood” Anal. Chem. 71:1138-1145) and many others. Similarly, integrated systems for performing mixing experiments, DNA amplification, DNA sequencing and the like are also available. See, e.g., Service (1998) “Coming Soon: the Pocket DNA Sequencer” Science 282: 399-401. A variety of automated system components are available, e.g., from Caliper Technologies (Hopkinton, Mass.), which utilize various Zymate systems, which typically include, e.g., robotics and fluid handling modules. Similarly, the common ORCA® robot, which is used in a variety of laboratory systems, e.g., for microtiter tray manipulation, is also commercially available, e.g., from Beckman Coulter, Inc. (Fullerton, Calif.). Similarly, commercially available microfluidic systems that can be used as system components in the present invention include those from Agilent technologies and the Caliper Technologies. Furthermore, the patent and technical literature includes numerous examples of microfluidic systems, including those that can interface directly with microwell plates for automated fluid handling.

Any of a variety of liquid handling and/or array configurations can be used in the systems herein. One common format for use in the systems herein is a microtiter plate, in which the array or liquid handler includes a microtiter tray. Such trays are commercially available and can be ordered in a variety of well sizes and numbers of wells per tray, as well as with any of a variety of functionalized surfaces for binding of assay or array components. Common trays include the ubiquitous 96 well plate, with 384 and 1536 well plates also in common use. Samples can be processed in such trays, with all of the processing steps being performed in the trays. Samples can also be processed in microfluidic apparatus, or combinations of microtiter and microfluidic apparatus.

In addition to liquid phase arrays, components can be stored in or analyzed on solid phase arrays. These arrays fix materials in a spatially accessible pattern (e.g., a grid of rows and columns) onto a solid substrate such as a membrane (e.g., nylon or nitrocellulose), a polymer or ceramic surface, a glass or modified silica surface, a metal surface, or the like. Components can be accessed, e.g., by hybridization, by local rehydration (e.g., using a pipette or other fluid handling element) and fluidic transfer, or by scraping the array or cutting out sites of interest on the array.

The system can also include detection apparatus that is used to detect allele information, using any of the approached noted herein. For example, a detector configured to detect real-time PCR products (e.g., a light detector, such as a fluorescence detector) or an array reader can be incorporated into the system. For example, the detector can be configured to detect a light emission from a hybridization or amplification reaction comprising an allele of interest, wherein the light emission is indicative of the presence or absence of the allele. Optionally, an operable linkage between the detector and a computer that comprises the system instructions noted above is provided, allowing for automatic input of detected allele-specific information to the computer, which can, e.g., store the database information and/or execute the system instructions to compare the detected allele specific information to the look up table.

Probes that are used to generate information detected by the detector can also be incorporated within the system, along with any other hardware or software for using the probes to detect the amplicon. These can include thermocycler elements (e.g., for performing PCR or LCR amplification of the allele to be detected by the probes), arrays upon which the probes are arrayed and/or hybridized, or the like. The fluid handling elements noted above for processing samples, can be used for moving sample materials (e.g., template nucleic acids and/or proteins to be detected) primers, probes, amplicons, or the like into contact with one another. For example, the system can include a set of marker probes or primers configured to detect at least one allele of one or more genes or linked loci associated with a metabolic syndrome, insulin resistance, obesity, high blood pressure, dyslipidemia, diabetes and/or myocardial infarction phenotype, such as those listed in Tables 1-14. The detector module is configured to detect one or more signal outputs from the set of marker probes or primers, or an amplicon produced from the set of marker probes or primers, thereby identifying the presence or absence of the allele.

The sample to be analyzed is optionally part of the system, or can be considered separate from it. The sample optionally includes e.g., genomic DNA, amplified genomic DNA, cDNA, amplified cDNA, RNA, amplified RNA, proteins, etc., as noted herein. In one aspect, the sample is derived from a mammal such as a human patient.

Optionally, system components for interfacing with a user are provided. For example, the systems can include a user viewable display for viewing an output of computer-implemented system instructions, user input devices (e.g., keyboards or pointing devices such as a mouse) for inputting user commands and activating the system, etc. Typically, the system of interest includes a computer, wherein the various computer-implemented system instructions are embodied in computer software, e.g., stored on computer readable media.

Standard desktop applications such as word processing software (e.g., Microsoft Word™ or Corel WordPerfect™) and database software (e.g., spreadsheet software such as Microsoft Excel™, Corel Quattro PrO™, or database programs such as Microsoft Access™ or Sequel™, Oracle™, Paradox™) can be adapted to the present invention by inputting a character string corresponding to an allele herein, or an association between an allele and a phenotype. For example, the systems can include software having the appropriate character string information, e.g., used in conjunction with a user interface (e.g., a GUI in a standard operating system such as a Windows, Macintosh or LINUX system) to manipulate strings of characters. Specialized sequence alignment programs such as BLAST can also be incorporated into the systems of the invention for alignment of nucleic acids or proteins (or corresponding character strings) e.g., for identifying and relating alleles.

As noted, systems can include a computer with an appropriate database and an allele sequence or correlation of the invention. Software for aligning sequences, as well as data sets entered into the software system comprising any of the sequences herein can be a feature of the invention. The computer can be, e.g., a PC (Intel x86 or Pentium chip-compatible DOS™, OS2™ WINDOWS™ WINDOWS NT™, WINDOWS95™, WINDOWS98™, WINDOWS2000, WINDOWSME, or LINUX based machine, a MACINTOSH™, Power PC, or a UNIX based (e.g., SUN™ work station or LINUX based machine) or other commercially common computer which is known to one of skill. Software for entering and aligning or otherwise manipulating sequences is available, e.g., BLASTP and BLASTN, or can easily be constructed by one of skill using a standard programming language such as Visualbasic, Fortran, Basic, Java, or the like.

### Methods of Identifying Modulators of Metabolic Syndrome Phenotypes

In addition to providing various diagnostic and prognostic markers for identifying metabolic syndrome phenotypes, the invention also provides methods of identifying modulators of metabolic syndrome, insulin resistance, obesity, high blood pressure, dyslipidemia, diabetes and/or myocardial infarction phenotype. In the methods, a potential modulator is contacted to a relevant protein (such as those encoded by the genes or loci in Tables 1-14, 17 and 18) or to a nucleic acid that encodes such a protein. An effect of the potential modulator on the gene or gene product is detected, thereby identifying whether the potential modulator modulates the underlying molecular basis for the metabolic syndrome, insulin resistance, obesity, high blood pressure, dyslipidemia, diabetes and/or myocardial infarction phenotype.

In addition, the methods can include, e.g., administering one or more putative modulator to an individual that displays a relevant phenotype and determining whether the putative modulator modulates the phenotype in the individual, e.g., in the context of a clinical trial or treatment. This, in turn, determines whether the putative modulator is clinically useful.

The gene or gene product that is contacted by the modulator can include any allelic form noted herein. Allelic forms, whether genes or proteins, that positively correlate to undesirable metabolic syndrome, insulin resistance, obesity, high blood pressure, dyslipidemia, diabetes and/or myocardial infarction phenotypes are preferred targets for modulator screening.

Effects of interest that can be screened for include: (a) increased or decreased expression of products of the genes listed in Tables 1-14, 17 and 18 in the presence of the modulator; (b) a change in the timing or location of expression of the genes listed in Tables 1-14, 17, and 18, and/or products thereof, in the presence of the modulator; (c) a change in localization of the proteins encoded by the genes or loci of Tables 1-14, 17 and 18 in the presence of the modulator.

The precise format of the modulator screen will, of course, vary, depending on the effect(s) being detected and the equipment available. Northern analysis, quantitative RT-PCR and/or array-based detection formats can be used to distinguish expression levels of genes noted above. Protein expression levels can also be detected using available methods, such as western blotting, ELISA analysis, antibody hybridization, BIAcore, or the like. Any of these methods can be used to distinguish changes in expression levels of the genes of Tables 1-14, 17. and 18, and/or products thereof, that result from a potential modulator.

Accordingly, one may screen for potential modulators of the genes listed in Tables 1-14, 17 and 18, or products thereof, for activity or expression. For example, potential modulators (small molecules, organic molecules, inorganic molecules, proteins, hormones, transcription factors, or the like) can be contacted to a cell comprising an allele of interest and an effect on activity or expression (or both) of products of the genes listed in Tables 1-14, 17 and 18, can be detected. For example, expression can be detected, e.g., via northern analysis or quantitative (optionally real time) RT-PCR, before and after application of potential expression modulators. Similarly, promoter regions of the various genes (e.g., generally sequences in the region of the start site of transcription, e.g., within 5 KB of the start site, e.g., 1 KB, or less e.g., within 500BP or 250BP or 100 BP of the start site) can be coupled to reporter constructs (CAT, beta-galactosidase, luciferase or any other available reporter) and can be similarly be tested for expression activity modulation by the potential modulator. In either case, the assays can be performed in a high-throughput fashion, e.g., using automated fluid handling and/or detection systems, in serial or parallel fashion. Similarly, activity modulators can be tested by contacting a potential modulator to an appropriate cell using any of the activity detection methods herein, regardless of whether the activity that is detected is the result of activity modulation, expression modulation or both. These assays can be in vitro, cell-based, or can be screens for modulator activity performed on laboratory animals such as knock-out transgenic mice comprising a gene of interest.

Biosensors for detecting modulator activity detection are also a feature of the invention. These include devices or systems that comprise a protein product of a gene or locus of Tables 1-14, 17 and 18 coupled to a readout that measures or displays one or more activity of the protein. Thus, any of the above described assay components can be configured as a biosensor by operably coupling the appropriate assay components to a readout. The readout can be optical (e.g., to detect cell markers or cell survival) electrical (e.g., coupled to a FET, a BIAcore, or any of a variety of others), spectrographic, or the like, and can optionally include a user-viewable display (e.g., a CRT or optical viewing station). The biosensor can be coupled to robotics or other automation, e.g., microfluidic systems, that direct contact of the putative modulators to the proteins of the invention, e.g., for automated high-throughput analysis of putative modulator activity. A large variety of automated systems that can be adapted to use with the biosensors of the invention are commercially available. For example, automated systems have been made to assess a variety of biological phenomena, including, e.g., expression levels of genes in response to selected stimuli (Service (1998) “Microchips Arrays Put DNA on the Spot” Science 282:396-399). Laboratory systems can also perform, e.g., repetitive fluid handling operations (e.g., pipetting) for transferring material to or from reagent storage systems that comprise arrays, such as microtiter trays or other chip trays, which are used as basic container elements for a variety of automated laboratory methods. Similarly, the systems manipulate, e.g., microtiter trays and control a variety of environmental conditions such as temperature, exposure to light or air, and the like. Many such automated systems are commercially available and are described herein, including those described above. These include various Zymate systems, ORCA® robots, microfluidic devices, etc. For example, the LabMicrofluidic Device® high throughput screening system (HTS) by Caliper Technologies, Mountain View, Calif. can be adapted for use in the present invention to screen for modulator activity.

In general, methods and sensors for detecting protein expression level and activity are available, including those taught in the various references above, including R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2^{nd} Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and Satinder Ahuja ed., Handbook of Bioseparations, Academic Press (2000). “Proteomic” detection methods, which detect many proteins simultaneously have been described and are also noted above, including various multidimensional electrophoresis methods (e.g., 2-d gel electrophoresis), mass spectrometry based methods (e.g., SELDI, MALDI, electrospray, etc.), or surface plasmon reasonance methods. These can also be used to track protein activity and/or expression level.

Similarly, nucleic acid expression levels (e.g., mRNA) can be detected using any available method, including northern analysis, quantitative RT-PCR, or the like. References sufficient to guide one of skill through these methods are readily available, including Ausubel, Sambrook and Berger.

Whole animal assays can also be used to assess the effects of modulators on cells or whole animals (e.g., transgenic knock-out mice), e.g., by monitoring an effect on a cell-based phenomenon, a change in displayed animal phenotype, or the like.

Potential modulator libraries to be screened for effects on the genes or loci of Tables 1-14, 17, 18, or product thereof. These libraries can be random, or can be targeted.

Targeted libraries include those designed using any form of a rational design technique that selects scaffolds or building blocks to generate combinatorial libraries. These techniques include a number of methods for the design and combinatorial synthesis of target-focused libraries, including morphing with bioisosteric transformations, analysis of target-specific privileged structures, and the like. In general, where information regarding structure of products of the genes or loci of Tables 1-14, 17 18, is available, likely binding partners can be designed, e.g., using flexible docking approaches, or the like. Similarly, random libraries exist for a variety of basic chemical scaffolds. In either case, many thousands of scaffolds and building blocks for chemical libraries are available, including those with polypeptide, nucleic acid, carbohydrate, and other backbones. Commercially available libraries and library design services include those offered by Chemical Diversity (San Diego, Calif.), Affymetrix (Santa Clara, Calif.), Sigma (St. Louis Mo.), ChemBridge Research Laboratories (San Diego, Calif.), TimTec (Newark, Del.), Nuevolution A/S (Copenhagen, Denmark) and many others.

Kits for treatment of a metabolic syndrome, insulin resistance, obesity, high blood pressure, dyslipidemia, diabetes and/or myocardial infarction phenotype can include a modulator identified as noted above and instructions for administering the compound to a patient to treat treatment metabolic syndrome, insulin resistance, obesity, high blood pressure, dyslipidemia, diabetes and/or myocardial infarction.

### Cell Rescue and Therapeutic Administration

In one aspect, the invention includes rescue of a cell that is defective in function of one or more endogenous genes or polypeptides encoded by the genes or loci of Tables 1-14, 17 and 18 (thus conferring the relevant phenotype of interest, e.g., metabolic syndrome, insulin resistance, obesity, high blood pressure, dyslipidemia, diabetes and/or myocardial infarction, etc.). This can be accomplished simply by introducing a new copy of the gene (or a heterologous nucleic acid that expresses the relevant protein), i.e., a gene having an allele that is desired, into the cell. Other approaches, such as homologous recombination to repair the defective gene (e.g., via chimeraplasty) can also be performed. In any event, rescue of function can be measured, e.g., in any of the assays noted herein. Indeed, this method can be used as a general method of screening cells in vitro for expression or activity of any gene or gene product of Tables 1-14, 17 or 18. Accordingly, in vitro rescue of function is useful in this context for the myriad in vitro screening methods noted above. The cells that are rescued can include cells in culture, (including primary or secondary cell culture from patients, as well as cultures of well-established cells). Where the cells are isolated from a patient, this has additional diagnostic utility in establishing which sequence of Tables 1-14, 17 or 18 is defective in a patient that presents with a relevant phenotype.

In another aspect, the cell rescue occurs in a patient, e.g., a human or veterinary patient, e.g., to remedy a metabolic defect. Thus, one aspect of the invention is gene therapy to remedy metabolic defects (or even simply to enhance metabolic phenotypes), in human or veterinary applications. In these applications, the nucleic acids of the invention are optionally cloned into appropriate gene therapy vectors (and/or are simply delivered as naked or liposome-conjugated nucleic acids), which are then delivered, optionally in combination with appropriate carriers or delivery agents. Proteins can also be delivered directly, but delivery of the nucleic acid is typically preferred in applications where stable expression is desired. Similarly, modulators of any metabolic defect that are identified by the methods herein can be used therapeutically.

Compositions for administration, e.g., comprise a therapeutically effective amount of the modulator, gene therapy vector or other relevant nucleic acid, and a pharmaceutically acceptable carrier or excipient. Such a carrier or excipient includes, but is not limited to, saline, buffered saline, dextrose, water, glycerol, ethanol, and/or combinations thereof. The formulation is made to suit the mode of administration. In general, methods of administering gene therapy vectors for topical use are well known in the art and can be applied to administration of the nucleic acids of the invention.

Therapeutic compositions comprising one or more modulator or gene therapy nucleic acid of the invention are optionally tested in one or more appropriate in vitro and/or in vivo animal model of disease, to confirm efficacy, tissue metabolism, and to estimate dosages, according to methods well known in the art. In particular, dosages can initially be determined by activity, stability or other suitable measures of the formulation.

Administration is by any of the routes normally used for introducing a molecule into ultimate contact with cells. Modulators of and/or nucleic acids that encode the genes and loci of Tables 1-14, 17 or 18 can be administered in any suitable manner, optionally with one or more pharmaceutically acceptable carriers. Suitable methods of administering such nucleic acids in the context of the present invention to a patient are available, and, although more than one route can be used to administer a particular composition, a particular route can often provide a more immediate and more effective action or reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention. Compositions can be administered by a number of routes including, but not limited to: oral, intravenous, intraperitoneal, intramuscular, transdermal, subcutaneous, topical, sublingual, or rectal administration. Compositions can be administered via liposomes (e.g., topically), or via topical delivery of naked DNA or viral vectors. Such administration routes and appropriate formulations are generally known to those of skill in the art.

The compositions, alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and the like. Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations of packaged nucleic acid can be presented in unit-dose or multi-dose sealed containers, such as ampules and vials.

The dose administered to a patient, in the context of the present invention, is sufficient to effect a beneficial therapeutic response in the patient over time. The dose is determined by the efficacy of the particular vector, or other formulation, and the activity, stability or serum half-life of the polypeptide which is expressed, and the condition of the patient, as well as the body weight or surface area of the patient to be treated. The size of the dose is also determined by the existence, nature, and extent of any adverse side-effects that accompany the administration of a particular vector, formulation, or the like in a particular patient. In determining the effective amount of the vector or formulation to be administered in the treatment of disease, the physician evaluates local expression, or circulating plasma levels, formulation toxicities, progression of the relevant disease, and/or where relevant, the production of antibodies to proteins encoded by the polynucleotides. The dose administered, e.g., to a 70 kilogram patient are typically in the range equivalent to dosages of currently-used therapeutic proteins, adjusted for the altered activity or serum half-life of the relevant composition. The vectors of this invention can supplement treatment conditions by any known conventional therapy.

For administration, formulations of the present invention are administered at a rate determined by the LD-50 of the relevant formulation, and/or observation of any side-effects of the vectors of the invention at various concentrations, e.g., as applied to the mass or topical delivery area and overall health of the patient. Administration can be accomplished via single or divided doses.

If a patient undergoing treatment develops fevers, chills, or muscle aches, he/she receives the appropriate dose of aspirin, ibuprofen, acetaminophen or other pain/fever controlling drug. Patients who experience reactions to the compositions, such as fever, muscle aches, and chills are premedicated 30 minutes prior to the future infusions with either aspirin, acetaminophen, or, e.g., diphenhydramine. Meperidine is used for more severe chills and muscle aches that do not quickly respond to antipyretics and antihistamines. Treatment is slowed or discontinued depending upon the severity of the reaction.

### EXAMPLES

The following examples illustrate, but do not limit the invention. One of skill will recognize a variety of non-critical parameters that can be modified to achieve essentially similar results.

### Example 1

The entire human genome was scanned to identify common polymorphisms using microarray technology platforms as described in U.S. Ser. No. 10/106,097, entitled “Methods for Genomic Analysis”, filed on Mar. 26, 2002, assigned to the same assignee as the present application; U.S. Ser. No. 10/284,444, entitled “Chromosome 21 SNPs, SNP Groups and SNP Patterns,” filed on Oct. 31, 2002, assigned to the same assignee as the present application; and 10/042,819, entitled “Whole Genome Scanning,” filed on Jan. 7, 2002, assigned to the same assignee as the present application, all of which are incorporated herein by reference. The microarrays are manufactured using a process adapted from semiconductor manufacturing to achieve cost effectiveness and high quality.

### Example 2

Polymorphisms identified in Example 1 were grouped into haplotype blocks and haplotype patterns using methods disclosed in U.S. Ser. Nos. 10/106,097, entitled “Methods for Genomic Analysis”, filed Mar. 26, 2002 (Attorney Docket 200/1005-10), incorporated herein by reference. Representative polymorphisms, haplotype blocks and haplotype patterns from an entire human chromosome (chromosome 21) are disclosed in, for example, Patil, N. et al, “Blocks of Limited Haplotype Diversity Revealed by High-Resolution Scanning of Human Chromosome 21” Science 294, 1719-1723 (2001) and the associated supplemental materials, incorporated herein by reference.

### Example 3

DNA from each individual in the case (individuals who display a metabolic syndrome phenotype) and control (individuals who do not display the metabolic syndrome phenotype) groups was purified by methods well known in the art. DNA was isolated from human blood using QIAGEN's PAXGene kit (part # 761115), quantified the purified DNA using PicoGreen and the TECAN SpectraFluor Plus plate reader. Each DNA sample was subjected to a “normalization” procedure to equilibrate the DNA concentrations of each DNA sample, and the normalized DNA was subsequently quantified using the same PicoGreen/TECAN procedure as was used to quantify the pre-normalization DNA.

### Example 4

The DNA samples were amplified using short-range PCR. Methods for short-range PCR are disclosed, for example, in U.S. patent application Ser. No. 10/341,832, filed Jan. 14, 2003, entitled “Apparatus and Methods for Selection PCR Primer Pairs.” Briefly, the PCRs were performed in 384-well plates containing primer pairs to which short-range PCR reaction buffer, 15 ng sample DNA template, dNTPs, and TITANIUM™ Taq DNA polymerase (Clontech Laboratories, Inc., Mountain View, Calif.) were added in a total volume of 6 μl. The PCR plates were sealed prior to PCR. The thermocycler program used for the PCR is identified in Table 15:

TABLE 15

| Step | Action |
|---|---|
| 1 | Incubate at 96° C. for 5 min |
| 2 | Incubate at 96° C. for 2 seconds |
| 3 | Incubate at 53° C. for 2 minutes |
| 4 | Go to [step] “2” (for 55 subsequent cycles) |
| 5 | Incubate at 50° C. for 5 minutes |
| 6 | Hold at 4° C. (or at −20° C. if the plates are to |
| | be stored for more than a week |

### Example 5

PCR products (4 μl of each) were pooled into 96 deep well plates such that each well of the 96 deep well plates contained PCR products from 48 different PCR reactions. The pooled PCR products were treated with shrimp alkaline phosphatase (SAP) at 37° C. for 30 minutes. The reactions were subsequently heated to 80° C. for 20 minutes before cooling to 4° C. The completed reactions were stored at −20° C.

The SAP-treated PCR products were then purified using a vacuum filter apparatus. The products were transferred to a 3K vacuum filter plate, which was placed on the vacuum manifold of the vacuum filter apparatus. Vacuum was applied to the filter plate at >20 cm Hg until all the samples were dried. To resuspend the PCR products, 60 μl of molecular biology grade water was added to each well of each filter plate and the filter plates were incubated for at least 30 minutes at room temperature. The plates were vortexed at low speed for one minute every 5 minutes. Optionally, the plates were incubated overnight at 4° C. to increase recovery of the PCR product. Subsequently, for each well in the filter plate, the water previously added was used to wash the filter membrane three times before transferring the purified PCR product to a well of a new multiwell plate.

To quantitate the purified PCR product, 4 μl of the purified PCR product was added to 196 μl of water in a well in a quantification plate. The quantification plate was read using a TECAN plate reader, which provides an optical density reading for each purified PCR product, from which the concentration of the purified PCR product was computed.

### Example 6

The purified PCR products were labeled with biotin in a thermocycler. In each well of a multiwell plate, 0.5 μg of purified PCR product was added to 3 μl labeling buffer, 0.5 μl of 400 U/μl TdT, and 1 μl of 0.5 mM biotin mix (ddUTP/dUTP) in a final volume of 35 μl. The labeling buffer contained 100 mM Tris-acetate (pH 7.5), 100 mM magnesium acetate, and 500 mM potassium acetate. The DNA labeling conditions are as follows: 37° C. for 90 minutes, followed by 99° C. for 10 minutes and finally holding at 4° C.

### Example 7

The labeled PCR products were applied to microarrays containing oligonucleotides complementary to the genomic DNA that was amplified. Both strands of the labeled PCR product were probed for polymorphisms using microarray oligonucleotide probes. Since there are generally two alleles for a given polymorphic locus, the microarray contained both alleles of the complementary oligonucleotides at each polymorphic position so that the labeled DNA could be screened for both alleles of a given polymorphism simultaneously. Minor allele frequencies that varied significantly between the case group and control group were characterized as being associated with related disease. Results were verified by genotyping additional independent samples for polymorphisms that were potentially associated with the case or control group based on the initial analysis.

Prior to application to a microarray, 5 μl of herring sperm DNA (10 mg/ml) and 24 μl of 40% formamide were added to each well containing labeled PCR product. The plates were sealed, briefly vortexed, and spun down at 1,000 rpm for 15 seconds. The plates were placed into a thermocycler and the following program was run: 99° C. for 10 minutes, followed by a 65° C. soak for no more than 5 minutes. When denaturation was complete, prewarmed hybridization buffer was added to each well, and the plates were sealed and kept at 65° C. until the samples were transferred to the microarrays. The hybridization solution contains 60 μl of 5M TMACL, 1 μl of Tris pH 7.8 (or 8.0), 1 μl of 1% Triton X-100, 1 μl of 5 nM b-948 control oligo, and 1 μl of 10 mg/ml herring sperm DNA. A total of 120 μl of the denatured PCR product was transferred to a microarray that had been warmed at 50° C. for 30 minutes. The microarray containing the denatured PCR product was placed in a 50° C. hybridization oven where it was rotated at 20 r.p.m. for 16 (+3) hours such that the pooled sample was allowed to flow freely over the microarray during the incubation.

### Example 8

After incubation (i.e., hybridization), the microarray was removed from the hybridization oven and the sample was removed and stored at −20° C. Then, the microarray was placed into a flow-cell and washed two times with 15 ml of 1×MES. The microarray was inverted several times to ensure that the wash solution moved freely over the surface of the microarray prior to removing the wash solution. Finally, the flow-cell was filled with a third 15 ml aliquot of 1×MES.

The microarray was stained with sequential treatments with three different stain solutions for 20-30 minutes each, with each stain treatment separated by a wash step. The three stain solutions were as follows. Stain 1 consisted of 13.8 ml of 1×MES/0.1% Triton X-100, 2.0 ml of acetylated BSA (20 mg/ml stock solution), and 80 μl of streptavidin stock protein (1 mg/ml stock solution). Stain 2 consisted of 14.0 ml of 1×MES/0.1% Triton X-100, 2.0 ml of acetylated BSA (20 mg/ml stock solution), and 40 μl of biotinylated anti-streptavidin antibody (0.5 mg/ml stock solution). Stain 3 consisted of 13.8 ml of 1×MES/0.1% Triton X-100, 2.0 ml of acetylated BSA (20 mg/ml stock solution), and 80 μl of streptavidin Cy-chrome stock protein (0.2 mg/ml stock solution).

Staining with the three stain solutions was followed with a post-stain high stringency wash. Solutions of 6×SSPE and 0.2×SSPE were prewarmed to 37° C. The 1×MES was removed from the microarray and the microarray was rinsed once with ˜15 ml of prewarmed 6×SSPE. The microarray was inverted several times to ensure that the 6×SSPE moved freely over the surface of the microarray before it was removed. The 6×SSPE wash was removed and fresh 6×SSPE was added to the wafer. The wafer was incubated at 37° C. for 30 minutes, and the 6×SSPE was subsequently removed. The microarray was rinsed three times with prewarmed 0.2×SSPE, before being filled with fresh prewarmed 0.2×SSPE and placed in a 37° C. convection oven for 45 minutes. The 0.2×SSPE was removed and room temperature 1×MES was added to the microarray. The microarray was then inverted several times before the 1×MES was removed. The 1×MES wash was repeated once. Finally, fresh 1×MES was added to the microarray, which was wrapped in foil prior to storage at 4° C. or scanning of the microarray.

### Example 9

On the same days the microarrays were stained and washed, they were scanned using an arc scanner. After scanning, the microarrays were removed from the scanner, wrapped in foil and stored at 4° C. The scan files generated by the scanner were then analyzed by software programs designed to interpret intensity data from microarrays. This software allowed discrimination of hybridization patterns that distinguished the case pools from the control pools. The data were analyzed according to the methods disclosed in the following U.S. patent applications, all of which are assigned to the assignee of the present application: U.S. patent application Ser. No. 10/970,761, filed Oct. 20, 2004, entitled “Analysis Methods and Apparatus for Individual Genotyping”; and U.S. patent application Ser. No. 11/173,809, filed Jul. 1, 2005, entitled “Algorithm for Estimating Accuracy of Genotype Assignment”.

### Example 10

This Example relates to a whole genome association and replication analysis for 14 metabolic syndrome phenotypes, including metabolic syndrome (Table 1), diabetes (Table 2), myocardial infarction (Table 3), hypertension (Table 4), body mass index (a measure of obesity) (Table 5), waist circumference (a measure of obesity) (Table 6), diastolic blood pressure (Table 7), systolic blood pressure (Table 8), HDL cholesterol (Table 9), triglycerides levels (Table 10), insulin levels (Tables 11 and 12), and homeostasis analysis (The Homeostasis Model Assessment (HOMA) estimates steady state beta cell function and insulin sensitivity as percentages of a normal reference population) (Tables 13 and 14).

### Overview

Metabolic syndrome is a combination of medical disorders that affect a large number of people in a clustered fashion. According to the statistics, the prevalence in the USA is calculated as being up to 25% of the population [American Heart Association. Metabolic Syndrome—Statistics. Available from: www(dot)americanheart(dot)org/downloadable/heart/1081492779297FS15META4(dot)pdf], the end result of which is to increase one's risk for cardiovascular disease and diabetes. It is characterized by a group of metabolic risk factors in one person. These factors in general include: (1) Abdominal obesity (excessive fat tissue in and around the abdomen); (2) Atherogenic dyslipidemia (blood fat disorders—elevated triglycerides, reduced HDL cholesterol and high LDL cholesterol—that foster plaque buildups in artery walls); (3) Elevated blood pressure; and (4) Insulin resistance or glucose intolerance (the body can't properly use insulin or blood sugar). The causes of metabolic syndrome are extremely complex and remain mostly unknown. To elucidate the genetic contribution to this medical disorder, a two-stage whole-genome genetic association and replication study was carried out with the goal of identifying and validating genetic determinants of metabolic syndrome and the various related metabolic syndrome phenotypes.

### Study Design

This multi-stage study was designed to discover and validate the genetic associations with metabolic syndrome or any of its components, or “metabolic syndrome phenotypes.” The DNA samples used in the study were collected from multiple sources. The genotyping strategy included 3 stages of SNP selection, with individuals from 3 populations ((Europeans, Indian Asians and Mexicans). In Phase I, genome-wide association scans were performed in 1005 European men and 1006 Indian Asian men from the London Life Sciences Population (LOLIPOP) study, UK. In Phase II, 1822 SNPs were genotyped in 859 UK European women, 1181 UK Indian Asian women, 968 Mexican men and 1560 Mexican women. In the final, third stage (Phase III) 32 SNPs were validated in 5968 European male and female subjects. Collection of each cohort was approved by the relevant Institutional Ethics Committees, and all subjects gave written informed consent.

European and Indian Asian Subjects Genotyped in Phases I and II

LOLIPOP is a cohort study of cardiovascular health in men and women registered with family practitioners in West London, recruited with a response rate of 62%. Characteristics of subjects are shown in Tables S1 and S2. Europeans were recruited if all 4 grandparents were born in the UK; Indian Asians if all 4 grandparents were born in the Indian Subcontinent. The assessment of participants was carried out by trained research nurses according to a standardized protocol. An interviewer-administered questionnaire was used to collect data on medical history, family history, current prescribed medication (verified from the practice computerized records), cardiovascular risk factors, and alcohol intake. Country of birth of participants, parents, and grandparents were recorded together with language and religion for assignment of ethnic subgroups.

Mexican Subjects Genotyped in Phase II

Mexican men and women were recruited by the Instituto Nacional de Ciencias Medicas y Nutrición in Mexico City, Mexico. Subjects were identified from outpatient lipid, internal medicine, diabetes, thyroid, irritable bowel, and osteoarthritis clinics, and from local factories. Characteristics of the genotyped subjects are shown in Table S2.

Europeans Genotyped in Phase III

Subjects were recruited as part of the TNT study (LaRosa, J. C. et al., N. Engl. J. Med. 352, 1425-35 (2005)), and consisted of males and females between 35 and 75 years of age, who had clinically evident CHD. CHD was defined here as having previous myocardial infarction, previous or current angina with objective evidence of atherosclerotic CHD, or a history of coronary revascularization. Characteristics of genotyped subjects are shown in Table S3.

Case/Control Criteria

The case status for metabolic syndrome (Table 1) was defined using the ATP III (Adult Treatment Panel III) of the NCEP (National Cholesterol Education Program) (2001, 2005) definition of metabolic syndrome [Expert Panel on Detection, Evaluation, and Treatment of High Blood Cholesterol in Adults. Executive Summary of The Third Report of The National Cholesterol Education Program (NCEP) Expert Panel on Detection, Evaluation, And Treatment of High Blood Cholesterol In Adults (Adult Treatment Panel III). JAMA 2001; 285: 2486-97; Grundy S M, et al. (2005) Diagnosis and Management of the Metabolic Syndrome. An American Heart Association/National Heart, Lung, and Blood Institute Scientific Statement. Arterioscler Thromb Vasc Biol. 25: 2243-2244] i.e. individuals with three or more of the following:

Central/abdominal obesity as measured by waist circumference [Men—Greater than 40 inches (102 cm); Women—Greater than 35 inches (88 cm)]

Fasting triglycerides greater than or equal to 150 mg/dL (1.69 mmol/L)

HDL cholesterol [Men—Less than 40 mg/dL (1.04 mmol/L); Women—Less than 50 mg/dL (1.29 mmol/L)]

Blood pressure greater than or equal to 130/85 mm Hg or on antihypertensive medications.

Fasting glucose greater than or equal to 110 mg/dL (6.1 mmol/L).

Controls were defined as those who do not meet the above criteria. These same criteria were used to independently determine case/control status for other metabolic syndrome phenotypes. Specifically, the case status for waist circumference (Table 6) for men was greater than 40 inches, and for women it was greater than 35 inches; the case status for triglycerides (Table 10) was a fasting triglyceride level greater than or equal to 150 mg/dL; the case status for HDL cholesterol (Table 9) for men was less than 40 mg/dL and for women it was less than 50 mg/dL; the case status for systolic blood pressure (Table 8) was a measure greater than or equal to 130 mm Hg; the case status for diastolic blood pressure (Table 7) was a measure greater than or equal to 85 mm Hg; and the case status for hypertension (Table 4) was a blood pressure measure greater than or equal to 130/85 mm Hg or the patient currently being prescribed an antihypertensive medication. Controls were defined as those who do not meet the above criteria for these metabolic syndrome phenotypes.

In addition, the case status for diabetes (Table 2) was a fasting plasma glucose level greater than or equal to 126 mg/dL; the case status for myocardial infarction (Table 3) was a previous incidence of a myocardial infarction; and the case status for body mass index (BMI) (Table 5) was a BMI measure of greater than 30 kg/m². Two additional metabolic syndrome phenotypes were related to insulin level and HOMA measurement. Case/control status for the insulin level phenotype (Tables 11 and 12) was determined using linear regression modeling to fit the reference allele defined phenotype measurement curve. The significance testing was based on the co-efficient of the SNP, and this significance testing was used to divide the populations into case and control groups. For the HOMA score phenotype (Tables 13 and 14), case/control status was determined as previously described (Wallace, et al. (2004) Diabetes Care 27(6): 1487-95). (The HOMA score estimates steady state beta cell function (% B) and insulin sensitivity (% S) as percentages of a normal reference population, and serves as a measure of insulin sensitivity.) For both of these phenotypes, the analyses were performed twice for two different populations. The first analysis compared case and control individuals, all of whom were not diagnosed as diabetic (so diabetics were excluded from this analysis) (Tables 11 and 13). The second analysis compared case and control individuals, all of whom were not Mexican (so the Mexican populations were excluded from this analysis) (Tables 12 and 14).

### Genotyping

Sample Preparation

Whole-genome amplification was performed on samples with less than 35 μg genomic DNA as described elsewhere (Bacann et al., Am. J. Hum. Genet. 66, 1933-1944 (2000)). Multiplex PCR reactions were set up as follows (per reaction): 10 ng of genomic DNA was amplified using ˜220-plex PCR primer pairs (0.1 μM of each primer), 3.25 mM dNTPs, 12.5× Titanium Taq (Clontech), 0.19M Tricine, 3.35× MasterAmp PCR Enhancer with Betaine (Epicentre Biotechnologies), 0.235% DMSO, 0.3M KCl, 0.37M Trizma, 0.1 M (NH₄)₂SO₄, and 0.02M MgCl₂, in a volume of 61. Thermocycling was performed using a 9700 cycler (Perkin-Elmer) as follows: 5 minutes at 96° C.; 55 cycles of 96° C. for 2 seconds and 53° C. for 2 minutes per cycle, then 15 minutes at 50° C. Excess unincorporated nucleotides were dephosphorylated using Shrimp Alkaline Phosphatase (SAP), and purified using a 3K 96-well filter plate (Pall Scientific) fitted onto a vacuum manifold with a pressure of >25 cm Hg. 16 μg of each purified pooled PCR product was labeled with 40 nmol each of biotin-16-ddUTP and biotin-16-dUTP (Perkin Elmer) using 1400 units of recombinant TdT (Roche). 7.4 μl of 10 mg/ml herring sperm DNA was added to each DNA sample and the samples denatured at 99° C. for 20 minutes.

Array Hybridization

The labeled PCR products were hybridized to the high-density oligonucleotide arrays at 50° C. overnight in the following conditions: 70 ng/μl DNA, 3M TMACl, 10 mM Tris pH 8.0, 0.01% Triton X-100, 0.05 nM control oligo, and 0.42 mg/ml herring sperm DNA. After a brief wash in 1×MES buffer, the arrays were then incubated with 5 μg/ml streptavidin (Sigma Aldrich) for 15 minutes at 25° C., followed by 1.25 μg/ml biotinylated anti-streptavidin antibody (Vector Laboratories) for 10 minutes at 25° C., and then 1 μg/ml streptavidin-Cy-chrome conjugate (Molecular Probes) for 15 minutes at 25° C. After a final wash in 0.2×SSPE for 30 minutes at 37° C. the arrays were scanned with a custom built confocal laser scanner to measure the Cy-chrome fluorescence of the hybridized labeled sample. The intensities of the perfect-match and mismatch alleles features were used to determine the genotypes of the SNPs.

Data Filtering

After quality filters were applied to the stage one data, there were and 221,658 and 190,220 SNPs with a call rate of at least 90% that were polymorphic in the European and Indian Asian scans, respectively. Tests for association were performed for a smaller set of 216774 SNPs in the European scan and 180,410 SNPs in the Indian Asian scan, with Hardy-Weinberg equilibrium P>0.001. These sets had average call rates of 98.9% and 98.6%, respectively. The arrays used in the European scan performed better because these SNPs were chosen from a set that had already performed well on similarly designed arrays, and were specifically chosen for optimal coverage in a panel of European ancestry.

For stage two, 922 SNPs were genotyped for triglycerides, and 900 for HDL cholesterol. Of these, 689 and 656 respectively were polymorphic and passed QC (call rate >90% and Hardy-Weinberg P>10-9). For each phenotype, we tested just the subset of SNPs that had been selected for genotyping based on prior data for that phenotype in the whole genome stages of the project. In stage three, the average call rate for the 32 SNPs was 98.2%.

Statistical Analysis

Genotypes were coded as allele counts (0, 1, 2) in linear regression models. This corresponds to fitting an additive model where each allele copy makes the same incremental contribution to the phenotype. Models included adjustment for age, alcohol (stages one and two), gender (stages two and three) and CHD status (stage one). Triglycerides and HDL cholesterol were transformed prior to analysis using either −1/sqrt of log transformation to remove skew. The primary test for association consisted of a comparison of the variance explained by the full model, versus variance explained by a model without the genotype term.

In the stage one scans, we did not see strong evidence for population stratification in the test results. Using Genomic Control (Bacanu et al., Am. J. Hum. Genet., 66, 1933-1944 (2000)) variance inflation factors determined for the various phenotypes were ≦1.07, so we did not make corrections for population structure.

In stage two, we used principal components analysis (PCA) to characterize population structure (Price, 2006, supra). The first two principal components effectively capture the reported ancestry of the samples (FIG. 5I). The third component identified a subset of SNPs whose genotypes were unusually sensitive to experimental variability in the genotyping process. The existence of this type of artifact and the ability of PCA to detect it has been previously noted (Price, 2006, supra, Clayton et al., Nat. Genet. 37, 1243-1246 (2005)). This component was correlated with variability in overall brightness of a microarray, which is sensitive to experimental variation in the fragmentation, hybridization, and staining processes. While the genotype calling algorithm attempts to correct for systematic scan-level variation, the correction is less successful for some SNPs. Additional components generally seemed to be associated with individual elements of local linkage disequilibrium structure, rather than correlations across unlinked markers. Hence we included just the top three components as covariates in the regression models.

To improve the accuracy of P values and false discovery rate estimates in stage two, we also employed genomic control to measure and eliminate limited amounts of residual variance inflation in the test statistics. For each phenotypic analysis, we computed variance inflation factors using the complementary set of SNPs genotypes in stage two that had not been selected specifically for association testing on that phenotype. We first transformed P values to χ2 statistics, and then computed the inflation factor as the median test statistic divided by 0.455. This yielded an inflation factor of 1.05 for triglycerides and 1.10 for HDL cholesterol. Residual variance inflation may be due to population structure not accounted for by the top principal components, or violations of assumptions of the parametric models (i.e. homoscedasticity, normality of residuals).

Stage three involved analysis of 32 SNPs in a cohort of men and women of European ancestry. Results of linear regression analyses in stage three were combined with results of stage two, using Fisher's method (Fisher R. A., Statistical methods for research workers 13^{th} ed., Oliver & Lloyd, London (1925)). This approach was used instead of a joint analysis, because we did not genotype a sufficient number of SNPs in stage 3 to model population structure in that set of samples. We did not include stage one data in the combined analysis as SNPs tested in stage two and three were not consistently present in the stage one scans.

For many of our reported associations, we identified multiple SNPs in the same genomic interval. To determine the extent to which these associations were independent, as opposed to indirect associations due to linkage disequilibrium, we performed analyses of two-SNP models in the stage two and stage three data. Given a pair of SNPs from Table 15 in the same genomic interval, we determined whether one SNP still accounted for a significant amount of variance, after conditioning on the other SNP. This analysis cannot prove independence, since two assayed markers may be in low LD with one another, but both be in LD with a hidden causal variant. But it can identify sets of markers consistent with just one causal variant. Since we did not attempt to obtain comprehensive coverage of common variants in these intervals, we think this pairwise analysis is more appropriate than a haplotype based approach.

In the triglyceride analysis, three regions were identified with multiple significant associations: the MLXIPL region with four SNPs, the LPL region with five SNPs, and the APO cluster region with four SNPs. For the MLXIPL region, conditioning on rs3812316 accounted for association at rs12056034, rs17145732, and rs799160 (stage two: P=0.83, P=0.27, P=0.35; stage three: P=0.13, P=0.14, P=0.03). For the LPL region, conditioning on rs328 accounted for association at rs325, rs17410914, and rs4406409 (P=0.57, P=0.07, P=0.15). There was weak evidence for residual association at rs326 (P=0.004) but rs328 still accounted for most of the variance attributable to rs326. For the APO region, conditioning on rs1558861 accounted for associations at rs2075292, rs7124741, and rs17120139 (stage two: P=0.10, P=0.17, P=0.15; stage three: P=0.17, P=0.10, P=00.08).

We examined the association of rs3812316 with other metabolic phenotypes. Although rs3812316 was associated in stage two with HDL (P=0.0002), hypertension (P=0.02) and metabolic syndrome (P=0.01), these relationships were confounded by the correlations of these phenotypes with triglycerides. After adjustment for triglycerides, the associations were no longer significant. In stage three, the association of rs3812316 with these phenotypes did not replicate.

The relationship of rs3812316 with triglycerides was not influenced by BMI or gender.

In the HDL analysis, two regions were identified with multiple significant associations: the LPL region with three SNPs, and the CETP region with six SNPs. For the LPL region, in stage two, tests for conditional association indicated that the three SNPs were not independent, but no SNP stood out as most informative (for all two-SNP models, P>0.05 for a second SNP effect). In stage three, rs326 was most informative, and conditioning on this SNP abolished associations at rs325 and rs328 (P=0.85, P=0.93). For the CETP region, in stage two, conditioning on rs7205804 accounted for associations at rs2217332, rs711752, and rs5882 (P=0.27, P=0.05, P=0.10). The signal at rs5880 was partially independent of rs7205804 (P=3.2×10−7), and rs1800777 was accounted for by rs5880 (P=0.89). In stage three, we saw a similar pattern, though in this case, rs711752 was best at accounting for rs2217332, rs7205804, and rs5882 (P=0.09, P=0.87, P=0.22).

Again, the signal at rs5880 was partially independent of rs711752 (P=3.7×10−8) and accounted for rs1800777 (P=0.19). Lipid lowering with statins may have influenced the genotype-phenotype relationships. Although we did not adjust for statin use in stages 1 and 2 (where usage averaged 37% and 10% respectively), measurements for stage 3 were taken after a “wash-out” period with no statin usage, and therefore are unaffected by treatment confounding.

To address the question of SNP interactions, for triglyceride and HDL analyses, we examined all two-SNP models for the SNPs identified in Table 1, in the stage two data. We included additive main effects for both SNPs plus a multiplicative interaction term. We assessed significance of the interaction term by analysis of variance after accounting for main effects. For both phenotypes, we found no interactions that were significant after Bonferroni correction. We repeated the analyses with genotypes coded as factors rather than allele counts, with the same result.

### Phase I

In the first phase, genome-wide association scans were performed on DNA from the male Indian Asian cases and controls for a total of 248,537 single nucleotide polymorphisms (SNPs) evenly spaced through out the human genome. Similarly, the male Caucasian case and control samples were genotyped on a total of 266,722 SNPs chosen to represent the maximum coverage based on linkage disequilibrium (LD) previously established in the European Caucasian population [David A. Hinds et al. (2005) Whole genome patterns of common DNA variation in three human populations. Science, Vol 307, p 1072-1079]. Rigorous quality control filters [David A. Flinds et al. (2004) Application of pooled genotyping to scan candidate regions for association with cholesterol levels. Human Genomics, Vol 1. No. 6, p 421-434] were applied to exclude allele frequency estimates that were determined to be unreliable. In the European and Indian Asian scans, 216,774 and 180,410 SNPs were successfully genotyped, respectively.

All of the samples used in Phase I were of male gender. The cases were selected based on the criteria described above and the controls were selected by random digit dialing recruitment matching the geographical location near London, U.K. Some of these subjects were on anti-diabetic or anti-hypertensive medications when samples were collected.

Primary analysis tested for association of SNPs with case/control status, based on the ATPIII definition. Secondary analyses were performed to identify the potential SNP markers that were associated with the individual components of the metabolic syndrome listed above. The two populations were analyzed separately and the results were used to pick SNPs for replication in additional populations.

Three SNPs showed genome-wide significance after Bonferroni correction. Two SNPs, rs1558861 and rs17120139, both flanking the APOA1-ApoC3-APOA4-APOA5 gene cluster on chromosome 11q23, were associated with triglycerides in the Indian Asian scan (P=3.8×10⁻⁵ and P=0.011, respectively, after correction for 180,410 tests). One SNP, es711752 in the CETP gene, was associated with HDL cholesterol in the European scan (P=7.2×10⁻⁵ after correction for 216,774 tests).

### Phase II

There were four distinct populations used for Phase II of the study: 1181 female Indian Asians, consisting of 407 cases and 774 controls; 859 female Caucasians, consisting of 153 cases and 707 controls; 1560 female Mexicans, consisting of 774 cases and 785 controls; and 969 male Mexicans, consisting of 402 cases and 567 controls.

The female Indian Asian and Caucasian samples were characterized in the same way as the male Indian Asian and Caucasian samples used in Phase I, although these female samples were collected predominantly by random digit dialing with some of them being based on cardiovascular disease status.

The Mexican samples were collected by the Instituto Nacional de Ciencias Medicas y Nutricion in Mexico City, Mexico. Control samples in this cohort consist of healthy individuals aged 40 or over without any chronic disorder with normal HDL cholesterol and normal blood pressure. Patients with hypothyroidism, irritable bowel syndrome, or osteoarthrosis can be included. The control samples were identified from patients of the thyroid clinic or those with irritable bowel disease or osteoarthritis. In addition, workers (aged 40 or older) in some factories were offered free medical consultation and the opportunity to participate in the study offered. Exclusions for controls included the use during the previous two months of beta blockers, diuretics, glucocorticoids, estrogen, progestins, androgens, and statins; a body mass index above 30 kg/m²; type 2 diabetes; CHD; and gestational diabetes.

The primary goal of the Phase II statistical analysis was to validate the SNPs selected for each outcome of interest. For each outcome, all four groups were fit using a single statistical model with population being treated as a factor with four levels (Mexican males, Mexican females, UK Caucasian females, UK Indian females). This model included a categorical variable indicating the source population (along with appropriate interactions with other covariates).

In this second (“replication”) phase, multiple strategies were used to identify SNPs for replication. To enhance power, in Phase II SNPs that showed equivocal significance in the genome-wide scans were selected for further testing in larger cohorts. Approximately one third of the SNPs were chosen based on those that were significantly associated with the most critical phenotypes (Metabolic syndrome, HOMA, HDL, BMI, coronary heart disease, and hypertension) with p<0.0001 in either scan or with p<0.001 and the direction of change the same between the Phase I scans as well as SNPs in druggable or extracellular genes with p<0.01 in any phenotype. Another one third was picked using multivariate analysis with the three major components of disease (lipids, glucose/insulin, and hypertension/blood pressure). The remaining one third of SNPs was chosen by the relevant therapeutic areas using a variety of criteria. There was overlap of SNPs among these categories. Additional SNPs were selected (from dbSNP) to enhance coverage in the vicinity of the identified associations. In this fashion, 922 SNPs were identified for further testing against triglyceride levels and 900 SNPs for further testing against HDL cholesterol in 4,568 individuals from four additional cohorts (859 European women, 1,181 Indian Asian women, 1,560 Mexican women and 968 Mexican men).

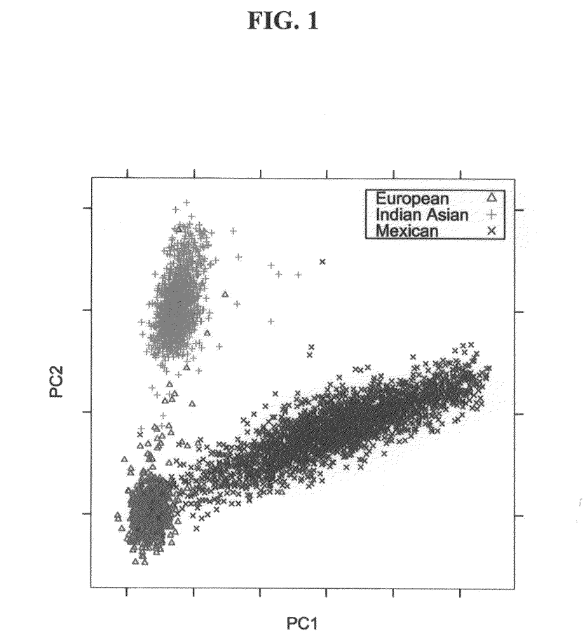

Genotyping was performed as described above, using a custom array, and was successful for 689 triglyceride and 656 HDL cholesterol SNPs, consistent with previous experience (Patil, L. et al. Science 294, 1719-1723 (2001). Data were analyzed as described above, under an additive model, using linear regression combined with principal components analysis to model population structure in the combined dataset (Price, A. L. et al., Nat. Genet. 38, 904-909 (2006)), assuming a common genotype effect across populations (see also FIG. 1). Genomic control was used to adjust the distributions of test statistics for residual variance inflation (Bacanu et al., 2000, supra).

### Phase III

In Phase III, the findings of Phase II were replicated, by genotyping all SNPs with false discovery rate <0.40. This comprised 22 SNPs for triglycerides and 15 for HDL cholesterol, which we tested using Taqman assays in 4,836 men and 1,132 women of European ancestry from the Treating to New Targets (TNT) study (LaRosa et al., N. Engl. J. Med. 352, 1425-1435 (2005)). Fisher's method was used to provide a combined analysis of the results from Phases II and III (Fisher, R. A., Statistical Methods for Research Workers, supra).

### Results

Replication genotyping results were generated for 4,982 SNPs that were polymorphic and had call rates of at least 80%, out of 5,716 SNPs attempted. When calculating false discovery rates, results for 369 SNPs that were substantially out of Hardy Weinberg equilibrium in both the UK Indian and Caucasian female samples (P<10⁻¹⁰), or that showed evidence of inconsistent genotyping across experiment dates (P<10⁻¹⁰) were excluded. For each phenotype, the regression analysis was done such that the p value was calculated against the complete subset of SNPs used in the replication genotyping. The p value cut-off of 0.05 was adopted for the replication test significance. False discovery rates (FDRs) across the complete subset of SNPs were also computed.

In summary, 2,348 single nucleotide polymorphisms (SNPs) tested positive for replication of significant (p≦0.05) associations with metabolic syndrome, or one of the related phenotype outcomes. The SNPs meeting the significance level and thereby identified as associated with one or more metabolic syndrome phenotypes are listed in Tables 1-14.

Tables 1-14 are individual worksheets for metabolic syndrome status or one of the components: metabolic syndrome case control status (Table 1); diabetic case control status (Table 2); myocardial infarction case control status (Table 3); hypertension case control status (Table 4); body mass index (BMI) (Table 5); waist circumference (Table 6); diastolic blood pressure (Table 7); systolic blood pressure (Table 8); HDL cholesterol (Table 9); triglycerides (Table 10); insulin: Depending on the availability of insulin measurement values, the analysis was subdivided into non-diabetic and non-Mexican analyses (Tables 11 and 12, respectively); and HOMA: Similarly as for insulin, the analysis of homeostasis analysis was subdivided into non-diabetic and non-Mexican analyses (Tables 13 and 14, respectively). The following is a description of the column headings for Tables 1-14.

TABLE 16
COLUMN IDENTIFIERS FOR TABLES 1-14

| Column | |
|---|---|
| Name | Description |
| snp_id | SNP identifier. Perlegen SNP identifiers may be used for |
| | accessing additional information about the SNP using the |
| | Genotype Browser on the Perlegen Sciences, Inc. website |
| | (genome(dot)perlegen(dot)com/browser/index.html). |
| refsnp_id | NCBI dbSNP identifier. |
| chrom | Chromosome ID (1-22, X or Y). |
| Accession | NCBI GenBank sequence accession and version number. |
| Position | SNP position in NCBI build 34 sequence. |
| Alleles | The allelic sequences for the SNP. |
| Flank | The two 25-mer sequences flanking the SNP. |
| genes_near | A text representation of genes surrounding the SNP. |
| m_freq | Frequency of the reference allele in the Mexican samples. |
| i_freq | Frequency of the reference allele in the UK Asian Indian |
| | samples. |
| c_freq | Frequency of the reference allele in the UK Caucasian |
| | samples. |
| p_freq | P value for significance of the genotype term(s). |
| q_freq | Estimated false discovery rate (FDR) for the P value |

Attached Table 18, also using some of the above identifiers, summarizes the results for SNPs that were subject to validation in the metabolic syndrome study described in the present example.

Further identifiers used in the columns of Table 18 are explained in Table 19. In Table 18 the “genes_near” column lists all transcripts within 50 kb, and at least one transcript upstream and downstream regardless of distance. A spacer like “- -” indicates an interval or more than 50 kb, and longer spacers indicate longer intervals. Transcripts that contain the SNP are enclosed in brackets.

For HDL and triglycerides, the measurements were log transformed, so the effect sizes from the linear regression are in log units. For hypertension and metabolic syndrome case/control status, logistic regression was used, and the effect sizes are in log odds units.

Cells in the stage 1a, 1b, and 3 results are colored to indicate consistency of those results with the stage 2 results. Results are colored green if they are consistent with the direction of effect in stage 2, or red if they are inconsistent. It is emphasized, however, that “poor correlation” in this data set should not be viewed as an indication that such correlation does not exist. The intensity is scaled based on the corresponding P value. The designation of positions and gene links in Table 18 has been updated in accordance with NCBI Build 36. For instance, the gene originally designated WBSCR14 is now referred to as MLXIPL.

Table 18 includes associations with HDL, triglycerides, hypertension status, and metabolic syndrome case/control status. As described above, stages 1a and 1b of this study were genome scans in Indian Asian and Caucasian men; stage 2 was replication in Mexican men and women, and Indian and Caucasian women; and stage 3 was replication in Caucasian men and women from the TNT study. SNPs were selected for validation based on a false discovery rate <0.4 in stage 2 of the study. Alleles are provided as reference over alternate sequence. It is noted that some SNPs listed in Table 18 have shown associations on multiple phenotypes:

HDL and triglycerides: rs17145732, rs325, rs326, rs328, rs4824743.

HDL and case/control status: rs7205804.

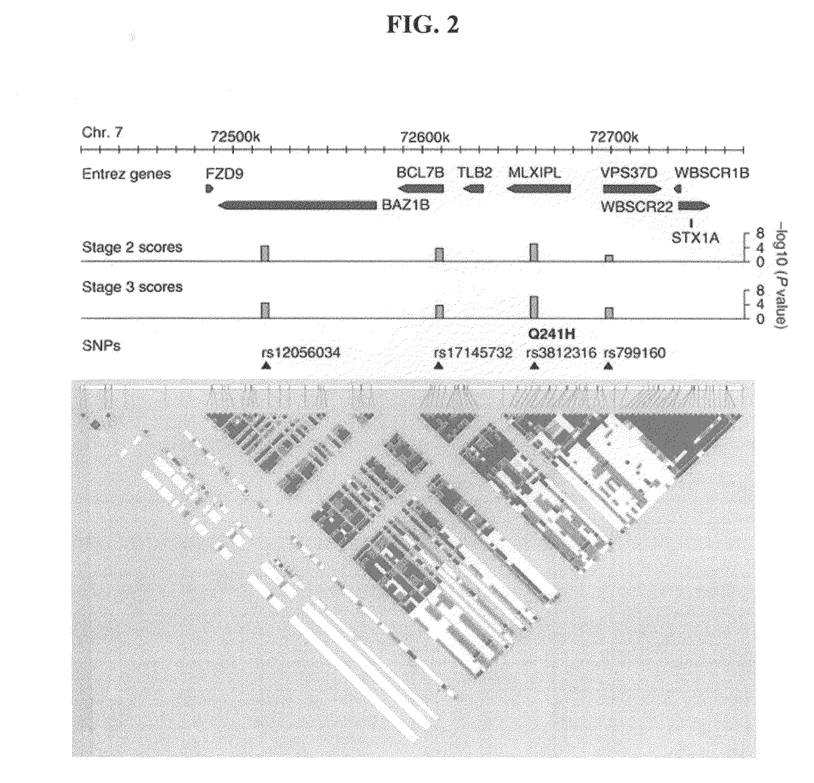

For triglycerides, 13 SNPs were found with combined P values significant at P<7×10⁻⁵ (equivalent to P<0.05 after Bonferroni correction for 689 successfully genotyped SNPs (Table 18). Four SNPs in the MLXIPL region were associated with raised triglyceride levels. These SNPs fall within an interval of high linkage disequilibrium spanning more than 200 kb (FIG. 2). The most significant association was a nonsynonymous (ns) SNP (rs3812316, Gln241His) in MLXIPL, with a combined uncorrected P value across Phases II and III of 0.4×10⁻¹⁰ (corresponding to P=9.9×10⁻⁸ after Bonferroni correction). SNP rs3812316 conferred an odds ratio of 1.29 per copy of the major C allele for triglyceride levels >1.7 mmol/l. Median triglyceride levels were 2.07, 1.996 and 1.75 mmol/l with rs3812316 CC, GC and GG genotypes, respectively, among subjects in Phase III, of which 80% were homozygous for the high-risk wild-type allele (Table 18). There was a similar gradation in triglyceride levels in the Phase II populations. There were no significant associations between rs3812316 and other tested phenotypes after adjusting for triglyceride levels and accounting for multiple testing, as described above. Regression analyses indicated that these results were consistent with a single casual variant within the MLXIPL region.

Of the remaining nine markers associated with triglycerides, four were in the vicinity (within 70 kb) of the APOA1-APOC3-APOA4-APOA5 cluster, and five were in or around the LPL gene. SNP rs3812316 in MLXIPL accounted for a similar proportion of population variation in triglyceride levels, compared to SNPs in LPL and the APO cluster. For HDL cholesterol, 9 SNPs were found with combined P<8×10⁻⁵ (equivalent to P<0.05 after Bonferroni correction for 656 successfully genotyped SNPs, Table 18). Six of these were in or around the CETP gene, one was in the LPL gene, one was in the LIPC gene and one was a nsSNP in ABCA1 (rs9282541, Arg230Cys) that was frequent only in the Mexican cohorts. All of these are well-recognized associations (Ordovas, J. M. Cardiovasc. Drugs. Ther. 16, 273-281 (2002), Romeo et al., Nat. Genet. 39, 513-516 (2007), Saxena et al., Science 316, 1331-1336 (2007)).

Although deep resequencing of the locus and functional studies will be needed to identify the casual variant, the finding of an association between nsSNP rs3812316 in MLXIPL and triglyceride levels in man is new and biologically compelling. MLXIPL is a transcription factor with a pivotal role in glucose utilization and energy storage (Uyeda & Repa, J. Cell. Metabolism 4, 107-110 (2006), Ma et al., J. Biol. Chem. 281, 28721-28730 (2006)). SNP rs3812316 is in an evolutionarily conserved region of the gene encoding a domain involved in glucose-dependent activation of MLXIPL (Li et al., Diabetes 55, 1179-1189 (2006)). Glucose flux into hepatocytes results in elevated xylulose 5-phosphate, which promotes the action of protein phosphatase 2A and results in nuclear translocation of MLXIPL. Nuclear MLXIPL dimerizes with MLX and binds carbohydrate response elements to increase transcription of genes involved in glycolysis, lipogenesis, triglyceride synthesis and very-low-density lipoprotein secretion. Conversely, starvation or high-fat as compared to high-carbohydrate feeding increases the activities of cAMP- and AMP-dependent kinases, respectively, and phosphorylate MLXIPL to reduce DNA binding and promote nuclear exclusion of MLXIPL>.

The low triglyceride levels associated with the Gln241His polymorphism suggest reduced MLXIPL function and are consistent with the low triglyceride levels in MLXIPL-null mice (Uyeda & Repa, J. Cell Metabolism 4, 107-110 (2006)). The major, wild-type Gln241 allele of MLXIPL is associated with increased triglyceride synthesis. Wild-type MLXIPL may thus permit more efficient food utilization, fat deposition and rapid weight gain at times of food abundance, making individuals better able to survive subsequent times of famine. In modern times, with continuous and reliable food supply, these genes may become disadvantageous, leading to excess energy storage in preparation for a famine that never comes. MLXIPL is therefore a plausible candidate for a “thrifty gene,”—the classic concept proposed by Neel in 1962 (Diamond, J., Nature 423, 599-602 (2003)).

### Example 11

### Background

Indian Asians (IA) are at increased risk of Type 2 Diabetes (T2D) compared to other populations. In the UK, T2D prevalence is 4 times higher among IA compared with Northern Europeans (NE). Recent studies in Caucasian populations have suggested an association of allelic variation in the TCF7L2 gene with T2D (Grant, et al. (2006) Nature Genetics 38(3):320-323). The purpose of this study was to i) replicate previous finding in NE and ii) investigate whether TCF7L2 is associated with T2D in IA, and accounts for their increased risk of T2D.

Methods

We investigated 1006 IA (271 T2D+, 735 T2D−), and 1005 NE (177 T2D+, 828 T2D−) men aged 35-65 yrs. Based on the LD structure of SNPs in the TCF7L2 interval in HapMap CEU samples, two SNP markers (rs4506565 and rs6585200) for which we had previously developed assays, and which showed moderate to strong pairwise r² with the reported SNP markers in TCF7L2 were identified. All participants were genotyped for rs4506565 and rs6585200.

Results

The allele frequencies in T2D cases and controls, and odds ratios for the association between genotype and T2D status are shown (Table 17). SNP rs4506565 was associated with T2D in both IA and NE, with both effects in the expected direction. There was no evidence of association between rs6585200 and T2D, however the LD data suggested a smaller effect at this site. There was no increase in the prevalence of either rs4506565 or rs6585200 amongst IA compared to NE.

### CONCLUSIONS

This study is the first to demonstrate that TCF7L2 is associated with T2D in IA, and further validates the association with T2D in Caucasian populations TCF7L2. As such, SNPs within or in linkage disequilibrium with TCF7L2 (in particular, SNP rs4506565) are useful for diagnosing or prognosticating type 2 diabetes, or determining the risk of developing type 2 diabetes, in individuals from Indian Asian and Caucasian populations.

TABLE 17

| | | | | |
|---|---|---|---|---|
| A/a | freq₁ | freq₀ | Odds Ratio [95% CI] | P value |

INDIAN ASIANS

| | | | | | |
|---|---|---|---|---|---|
| rs4506565 | T/_{A} | 0.359 | 0.294 | 1.34 [1.08, 1.66] | 0.0088 |
| rs6585200 | G/_{A} | 0.456 | 0.427 | 1.14 [0.93, 1.39] | 0.2132 |

Northern Europeans

| | | | | | |
|---|---|---|---|---|---|
| rs4506565 | T/_{A} | 0.370 | 0.302 | 1.29 [1.01, 1.64] | 0.0422 |
| rs6585200 | G/_{A} | 0.477 | 0.447 | 1.08 [0.85, 1.37] | 0.5181 |

Although the above discussion has presented the present invention according to specific methods, systems and apparatus, the present invention has a broader range of applicability. Further, while the foregoing invention has been described in some detail for purposes of clarity and understanding, it will be clear to one skilled in the art from a reading of this disclosure that various changes in form and detail can be made without departing from the true scope of the invention. For example, all the methods, techniques, systems, devices, kits, apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

Lengthy table referenced here
US20090186347A1-20090723-T00001
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20090186347A1). An electronic copy of the table will also be available from the USPTO upon request and payment of the fee set forth in 37 CFR 1.19(b)(3).

What is claimed is:

1. A method of identifying a metabolic syndrome phenotype for an organism, the method comprising:
detecting, in a biological sample from the organism, a polymorphism of a gene or a locus closely linked thereto, the gene selected from those listed in Tables 1-14, 17 and 18, wherein the polymorphism is associated with the metabolic syndrome phenotype; and,
correlating the polymorphism to the metabolic syndrome phenotype, thereby identifying the metabolic syndrome phenotype.

2. The method of claim 1, wherein the metabolic syndrome phenotype comprises a diagnosis of or predisposition to metabolic syndrome, insulin resistance, high blood pressure, dyslipidemia, diabetes, myocardial infarction or obesity.

3. The method of claim 2 wherein the metabolic syndrome phenotype is altered triglyceride levels.

4. The method of claim 3 wherein the polymorphism is in the MLXIPL region on chromosome 7 at 7q11.23.

5. The method of claim 4 wherein the polymorphism is in or proximal to a gene located in the MLXIPL region on chromosome 7 at 7q11.23, listed in Table 18.

6. The method of claim 3 wherein the metabolic syndrome phenotype is higher plasma triglyceride levels.

7. The method of claim 6 wherein the polymorphism is a single nucleotide polymorphism (SNP) in the MLXIPL gene or a gene or a locus closely linked thereto.

8. The method of claim 6 wherein the polymorphism is an allele at a SNP selected from the group consisting of: a cytosine at position rs1375388, an adenine at rs1448972, a guanine at rs 6844155, a cytosine at rs4960288, an adenine at rs 12056034, a thymine at rs 17145732, a cytosine at rs 3812316, an adenine at rs799160, a thymidine at rs325, an adenine at rd326, a cytosine at rs328, a cytosine at rs17410914, a thymidine at rs4406409, a cytosine at rs1558861, a guanine at rs2075292, an adenine at rs7124741, an adenine at rs17120139, a cytosine at rs9508032, a cytosine at rs9513115, a cytosine at rs9895521, a cytosine at rs747398, an adenine at rs4824743.

9. The method of claim 3 wherein the polymorphism is in the vicinity of the APOA1-APOA3-APOA4-APOA5 cluster.

10. The method of claim 9 wherein the polymorphism is at or proximal to a gene listed in Table 18.

11. The method of claim 3 wherein the polymorphism is a single nucleotide polymorphism (SNP) in the LPL gene or a gene or a locus closely linked thereto.

12. The method of claim 10 or 11 wherein the polymorphism is in a gene listed in Table 18.

13. The method of claim 2 wherein the metabolic syndrome phenotype is lower high density lipoprotein levels.

14. The method of claim 13 wherein the polymorphism is an ellele at a single nucleotide polymorphism (SNP) selected from the group consisting of: a thymidine at rs2992753, an adenine at rs2819770, a thymine at rs17145732, a thymine at rs325, an adenine at rs326, a cytosine at rs328, a thymine at rs9282541, a guanine at rs11858164, a thymine at rs2217332, a guanine at rs711752, a guanine at rs7205804, a cytosine at rs5880, an adenine at rs5882, an adenine at rs1800777, and an adenine at rs4824743.

15. The method of claim 2 wherein the metabolic syndrome phenotype is altered blood pressure.

16. The method of claim 15 wherein the metabolic syndrome phenotype is high blood pressure.

17. The method of claim 16 wherein the polymorphism is a guanine at rs5174.

18. The method of claim 2 wherein the metabolic syndrome phenotype is susceptibility to metabolic syndrome.

19. The method of claim 18 wherein the polymorphism is a guanine at rs1354746 or a guanine at rs7205804.

20. The method of claim 1, wherein the organism is a mammal, or the biological sample is derived from a mammal.

21. The method of claim 1, wherein the organism is a human patient, or the biological sample is derived from a human patient.

22. The method of claim 1, wherein the detecting comprises amplifying the polymorphism or a sequence associated therewith and detecting the resulting amplicon.

23. The method of claim 22, wherein the amplifying comprises:
a) admixing an amplification primer or amplification primer pair with a nucleic acid template isolated from the organism or biological sample, wherein the primer or primer pair is complementary or partially complementary to at least a portion of the gene or closely linked polymorphism, or to a proximal sequence thereto, and is capable of initiating nucleic acid polymerization by a polymerase on the nucleic acid template; and,
b) extending the primer or primer pair in a DNA polymerization reaction comprising a polymerase and the template nucleic acid to generate the amplicon.

24. The method of claim 22, wherein the amplicon is detected by a process that includes one or more of: hybridizing the amplicon to an array, digesting the amplicon with a restriction enzyme, or real-time PCR analysis.

25. The method of claim 22, comprising partially or fully sequencing the amplicon.

26. The method of claim 22, wherein the amplifying comprises performing a polymerase chain reaction (PCR), reverse transcriptase PCR (RT-PCR), or ligase chain reaction (LCR) using nucleic acid isolated from the organism or biological sample as a template in the PCR, RT-PCR, or LCR.

27. The method of claim 1, wherein the polymorphism is a SNP.

28. The method of claim 1, wherein the polymorphism comprises an allele selected from the group consisting of those listed in Tables 1-14, 17 or 18.

29. The method of claim 1, wherein the closely linked locus is about 5 cM or less from the gene.

30. The method of claim 1, wherein correlating the polymorphism comprises referencing a look up table that comprises correlations between alleles of the polymorphism and the phenotype.

31. The method of claim 1, wherein the organism is a non-human mammal and the method further comprises selecting the non-human mammal from a population of non-human mammals, based upon the phenotype.

32. The method of claim 31, comprising breeding the resulting selected non-human mammal with another non-human mammal to optimize the phenotype in one or more offspring.

33. A method of identifying a modulator of a metabolic syndrome phenotype, the method comprising:
contacting a potential modulator to a gene or gene product, wherein the gene or gene product is encoded by a gene or locus listed in Tables 1-14, 17 or 18; and,
detecting an effect of the potential modulator on the gene or gene product, thereby identifying whether the potential modulator modulates the metabolic syndrome phenotype.

34. The method of claim 33, wherein the metabolic syndrome phenotype comprises a diagnosis of or predisposition to metabolic syndrome, insulin resistance, high blood pressure, dyslipidemia, diabetes, myocardial infarction or obesity.

35. The method of claim 33, wherein the gene or gene product comprises a polymorphism selected from those listed in Tables 1-14, 17 or 18.

36. The method of claim 33, wherein the effect is increased or decreased expression of a gene or gene product encoded by one or more genetic loci listed in Tables 1-14, 17 or 18 in the presence of the modulator.

37. A kit for treatment of a metabolic syndrome phenotype, the kit comprising a modulator identified by the method of claim 33 and instructions for administering the compound to a patient to treat the metabolic syndrome phenotype.

38. The kit of claim 37, wherein the metabolic syndrome phenotype comprises a diagnosis of or predisposition to metabolic syndrome, insulin resistance, high blood pressure, dyslipidemia, diabetes, myocardial infarction or obesity.

39. A system for identifying a metabolic syndrome phenotype for an organism or biological sample derived therefrom, the system comprising:
a) a set of marker probes or primers configured to detect at least one allele of one or more gene or linked locus associated with the metabolic syndrome phenotype, wherein the gene or linked locus is one of those listed in Tables 1-14, 17 or 18;
b) a detector that is configured to detect one or more signal outputs from the set of marker probes or primers, or an amplicon produced from the set of marker probes or primers, thereby identifying the presence or absence of the allele; and,
c) system instructions that correlate the presence or absence of the allele with the predicted metabolic syndrome phenotype, thereby identifying the metabolic syndrome phenotype for the organism or biological sample derived therefrom.

40. The system of claim 39, wherein the metabolic syndrome phenotype comprises a diagnosis of or predisposition to metabolic syndrome, insulin resistance, high blood pressure, dyslipidemia, diabetes, myocardial infarction or obesity.

41. The system of claim 39, wherein the set of marker probes comprises a nucleotide sequence provided in Tables 1-14, 17 or 18.

42. The system of claim 39, wherein the detector detects at least one light emission, wherein the light emission is indicative of the presence or absence of the allele.

43. The system of claim 39, wherein the instructions comprise at least one look-up table that includes a correlation between the presence or absence of the allele and the metabolic syndrome phenotype.

44. The system of claim 39, wherein the system comprises a sample.

45. The system of claim 44, wherein the sample comprises genomic DNA, amplified genomic DNA, cDNA, amplified cDNA, RNA, or amplified RNA.

46. The system of claim 44, wherein the sample is derived from a mammal.

47. A method of predicting if an individual is at risk of developing a metabolic syndrome phenotype comprising:
a) genotyping said individual at genetic loci that are genetically linked to at least two genes associated with said metabolic syndrome phenotype; and
b) determining if the genotypes generated in a) comprise alleles that positively correlate to susceptibility to said metabolic syndrome phenotype, wherein if said alleles do positively correlate to susceptibility to said metabolic syndrome phenotype then said individual is determined to be at risk of developing said metabolic syndrome phenotype.

48. The method of claim 47, wherein the metabolic syndrome phenotype is metabolic syndrome and the at least two genes are listed in Table 1.

49. The method of claim 47, wherein the metabolic syndrome phenotype is diabetes and the at least two genes are genes listed in Table 2.

50. The method of claim 47, wherein the metabolic syndrome phenotype is myocardial infarction and the at least two genes are listed in Table 3.

51. The method of claim 47, wherein the metabolic syndrome phenotype is hypertension and the at least two genes are listed in Table 4.

52. The method of claim 47, wherein the metabolic syndrome phenotype is obesity and the at least two genes are listed in Table 5 and/or Table 6.

53. The method of claim 47, wherein the metabolic syndrome phenotype is high diastolic blood pressure and the at least two genes are listed in Table 7.

54. The method of claim 47, wherein the metabolic syndrome phenotype is high systolic blood pressure and the at least two genes are listed in Table 8.

55. The method of claim 47, wherein the metabolic syndrome phenotype is low HDL levels and the at least two genes are listed in Table 9.

56. The method of claim 47, wherein the metabolic syndrome phenotype is high fasting triglyceride levels and the at least two genes are listed in Table 10.

57. The method of claim 47, wherein the metabolic syndrome phenotype is high insulin levels and the at least two genes are listed in Table 11 and/or Table 12.

58. The method of claim 47, wherein the metabolic syndrome phenotype is insulin resistance and the at least two genes are listed in Table 13 and/or Table 14.

59. The method of claim 47, wherein said determining comprises referencing a look up table containing results from an association study, said association study comprising comparing allele frequencies for said genetic loci from individuals in a case group to allele frequencies for said genetic loci from individuals in a control group, wherein said results identify at least one allele that is more frequent in said case group than in said control group positively correlating to susceptibility to said metabolic syndrome phenotype.

60. A method of identifying a modulator of a metabolic syndrome phenotype, the method comprising administering a modulator of a gene or gene product, wherein the gene or gene product is encoded by a gene or locus listed in Tables 1-14, 17 or 18 to a non-human mammal or ex vivo mammalian cell, and measuring an effect indicative of a metabolic syndrome phenotype.

61. The method of claim 60, wherein the metabolic syndrome phenotype is obesity, hypertension, atherogenic dyslipidemia, diabetes, abnormal insulin levels, insulin resistance, glucose intolerance, risk of myocardial infarction, chronic prothrombotic state, or chronic proinflammatory state.

62. A method of identifying a metabolic syndrome phenotype for an organism, the method comprising:
detecting, in a biological sample from the organism, a haplotype in a genomic region comprising an allele selected from the alleles listed in Tables 1-14, 17 or 18; and
correlating the haplotype to the metabolic syndrome phenotype, thereby identifying the metabolic syndrome phenotype.

63. A method for identifying a human subject at increased risk for a metabolic syndrome phenotype, comprising using an in vitro assay to detect the presence of a risk allele provided in Table 18 in a human subject that is more frequently present in a population of humans with the metabolic syndrome phenotype than in a population of humans that do not have the metabolic syndrome phenotype, wherein the presence of the risk allele indicates that the human subject has an increased risk for the metabolic syndrome phenotype.

64. The method of claim 63 wherein the metabolic syndrome phenotype is high triglyceride levels and the risk allele is selected from the group consisting of a cytosine at position rs1375388, an adenine at rs1448972, a guanine at rs6844155, a cytosine at rs4960288, an adenine at rs12056034, a thymine at rs17145732, a cytosine at rs3812316, an adenine at rs799160, a thymine at rs325, an adenine at rs326, a cytosine at rs328, a cytosine at rs17410914, a thymine at rs4406409, a cytosine at rs1558861, a guanine at rs2075292, an adenine at rs7124741, an adenine at rs17120139, a cytosine at rs9508032, a cytosine at rs9513115, a cytosine at rs9895521, a cytosine at rs747398, and an adenine at rs4824743.

65. The method of claim 63 wherein the metabolic syndrome phenotype is lower high density lipoprotein levels and the risk allele is selected from the group consisting of a thymine at rs2992753, an adenine at rs2819770, a thymine at rs17145732, a thymine at rs325, an adenine at rs326, a cytosine at rs328, a thymine at rs9282541, a guanine at rs11858164, a thymine at rs2217332, a guanine at rs711752, a guanine at rs7205804, a cytosine at rs5880, an adenine at rs5882, an adenine at rs1800777, and an adenine at rs4824743.

66. The method of claim 63 wherein the metabolic syndrome phenotype is high blood pressure and the risk allele is a guanine at rs5174.

67. The method of claim 63 wherein the metabolic syndrome phenotype is metabolic syndrome and the risk allele is a guanine at rs1354746 or rs7205804.

68. A method for identifying a human subject at increased risk for coronary heart disease, comprising using an in vitro assay to detect the presence of a polymorphism with a linkage disequilibrium of at least r²=0.8 with a risk allele provided in Table 18 in a human subject, wherein the presence of the polymorphism indicates that the human subject has an increased risk for coronary heart disease.

69. The method of claim 63, wherein detecting the risk allele comprises detecting a single nucleotide polymorphism (SNP) allele.

70. A method for determining whether a human subject is at increased risk for a metabolic syndrome phenotype, comprising using an in vitro assay to detect the presence of a haplotype comprising a risk allele provided in table 19 in a human subject, wherein the presence of the haplotype indicates that the human subject has an increased risk for the metabolic syndrome phenotype.

71. The method of claim 70, wherein the metabolic syndrome phenotype is high triglyceride levels and the haplotype comprises one or more of the following: a cytosine at position rs1375388, an adenine at rs1448972, a guanine at rs6844155, a cytosine at rs4960288, an adenine at rs12056034, a thymine at rs17145732, a cytosine at rs3812316, an adenine at rs799160, a thymine at rs325, an adenine at rs326, a cytosine at rs328, a cytosine at rs17410914, a thymine at rs4406409, a cytosine at rs1558861, a guanine at rs2075292, an adenine at rs7124741, an adenine at rs17120139, a cytosine at rs9508032, a cytosine at rs9513115, a cytosine at rs9895521, a cytosine at rs747398, and an adenine at rs4824743.

72. The method of claim 70, wherein the metabolic syndrome phenotype is lower high density lipoprotein levels and the haplotype comprises one or more of the following: a thymine at rs2992753, an adenine at rs2819770, a thymine at rs17145732, a thymine at rs325, an adenine at rs326, a cytosine at rs328, a thymine at rs9282541, a guanine at rs11858164, a thymine at rs2217332, a guanine at rs711752, a guanine at rs7205804, a cytosine at rs5880, an adenine at rs5882, an adenine at rs1800777, and an adenine at rs4824743.

73. The method of claim 70, wherein the metabolic syndrome phenotype is high blood pressure and the haplotype comprises a guanine at rs5174.

74. The method of claim 70, where in the metabolic syndrome phenotype is metabolic syndrome and the haplotype comprises either a guanine at rs1354746 or a guanine at rs7205804.

75. The method of claim 63, wherein the human subject is heterozygous for the risk allele.

76. The method of claim 70, wherein the human subject is heterozygous for the haplotype.

77. A method for identifying a human subject at increased risk for a metabolic syndrome phenotype, comprising using an in vitro assay to detect the genotype of a SNP provided in Table 18, wherein the genotype of the SNP indicates that the human subject has an increased risk for the metabolic syndrome phenotype.

78. The method of claim 77, wherein the metabolic syndrome phenotype is high triglyceride levels and the genotype of the SNP is selected from the group consisting of: a cytosine at position rs1375388, an adenine at rs1448972, a guanine at rs6844155, a cytosine at rs4960288, an adenine at rs12056034, a thymine at rs17145732, a cytosine at rs3812316, an adenine at rs799160, a thymine at rs325, an adenine at rs326, a cytosine at rs328, a cytosine at rs17410914, a thymine at rs4406409, a cytosine at rs1558861, a guanine at rs2075292, an adenine at rs7124741, an adenine at rs17120139, a cytosine at rs9508032, a cytosine at rs9513115, a cytosine at rs9895521, a cytosine at rs747398, and an adenine at rs4824743.

79. The method of claim 77, wherein the metabolic syndrome phenotype is lower high density lipoprotein levels and the genotype of the SNP is selected from the group consisting a thymine at rs2992753, an adenine at rs2819770, a thymine at rs17145732, a thymine at rs325, an adenine at rs326, a cytosine at rs328, a thymine at rs9282541, a guanine at rs11858164, a thymine at rs2217332, a guanine at rs711752, a guanine at rs7205804, a cytosine at rs5880, an adenine at rs5882, an adenine at rs1800777, and an adenine at rs4824743.

80. The method of claim 77, wherein the metabolic syndrome phenotype is high blood pressure and the genotype of the SNP is a guanine at rs5174.

81. The method of claim 77, wherein the metabolic syndrome phenotype is metabolic syndrome and the genotype of the SNP is either a guanine at rs1354746 or a guanine at rs7205804.

82. A kit comprising one or more components for detecting the presence of a risk allele provided in Table 18 in a human subject.
